(19) United States
(12) Patent Application Publication
(10) Pub. No.: US 2024/0318244 A1
(43) Pub. Date: Sep. 26, 2024

(54) CLICK-CHEMISTRY BASED BARCODING

(71) Applicant: Bio-Rad Laboratories, Inc., Hercules, CA (US)

(72) Inventors: Man CHENG, Danville, CA (US); Jon PETERSEN, Santa Clara, CA (US)

(21) Appl. No.: 18/605,670

(22) Filed: Mar. 14, 2024

Related U.S. Application Data
(60) Provisional application No. 63/452,640, filed on Mar. 16, 2023.

(51) Int. Cl. C12Q 1/6869; C12N 15/10; C12Q 1/26; C12Q 1/34; C12Q 1/48; C12Q 1/6804; C12Q 1/6806; C12Q 1/6834; C12Q 1/6855

(52) U.S. Cl. CPC C12Q 1/6869; C12N 15/1096; C12Q 1/26; C12Q 1/34; C12Q 1/485; C12Q 1/6804; C12Q 1/6806; C12Q 1/6834; C12Q 1/6855; G01N 2333/90245; G01N 2333/91097; G01N 2333/9125; G01N 2333/978

(57) ABSTRACT

Methods and compositions for nucleotide sequencing are provided. In some embodiments, click chemistry is used to link barcoding oligonucleotides to DNA fragments comprising adapters introduced by a transposase.

### CROSS-REFERENCE TO RELATED PATENT APPLICATIONS

The present application claims benefit of priority to U.S. Provisional Patent Application No. 63/452,640, filed Mar. 16, 2023, which is incorporated by reference for all purposes.

### SEQUENCE LISTING

The instant application contains a Sequence Listing which has been submitted electronically in XML format and is hereby incorporated by reference in its entirety. Said XML copy, created on Apr. 23, 2024, is named 094868-1425407_121210US_SL.xml and is 6,331 bytes in size.

### BACKGROUND OF THE INVENTION

Tagging biological substrates with molecular barcodes in partitions can provide novel biological insight of the substrates that co-localize to discrete partitions, through the sequencing of the molecular barcodes and analysis, thereof. Increasing the number of barcoding competent partitions, such as droplets, increases the number of sequencing-based data points and converts a greater fraction of input substrates into data. Barcodes can be delivered to partitions, such as droplets, using beads as the delivery vehicle. In order to uniquely identify each partition, the beads can be labeled with clonal copies of unique barcode sequences, which can be released into the partition to tag molecules in the partition in a partition-specific manner.

### BRIEF SUMMARY OF THE INVENTION

In some embodiments, a method of nucleotide sequencing is provided. In some embodiments, the method comprises
forming cell reaction (i) hydrogel beads or (ii) semi-permeable capsules (SPCs) comprising single cells;
lysing the cells in the cell reaction hydrogel beads or SPCs such that at least a majority of nucleic acids of the cells is retained in the cell reaction hydrogel beads or SPCs, wherein the nucleic acids are DNA from the cell or RNA, and optionally converting the RNA into DNA with a reverse transcriptase;
contacting the DNA of the cells in the cell reaction hydrogel beads or SPCs with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked DNA fragment having the 5′ alkyne moieties;
partitioning in microwells the cell reaction hydrogel beads or SPCs comprising the DNA fragments with a barcoding hydrogel bead linked to barcoding oligonucleotides comprising (i) a barcode sequence that identifies the barcoding hydrogel bead and (ii) a 3′ azide moiety, thereby forming microwells containing one of the cell reaction hydrogel beads or SPCs and one of the barcoding hydrogel beads;
dissolving the cell reaction hydrogel beads or SPCs and barcoding hydrogel beads in the microwells;
after the dissolving, linking the 5′ alkyne moieties of the adaptor-linked DNA fragments to the 3′ azide moiety of the barcoding oligonucleotides via click chemistry to form a first and second barcoded strand of barcoded double-stranded DNA fragments,
recovering barcoded DNA fragments from the microwells and forming a mixture of barcoded DNA fragments from different microwells; and
performing nucleotide sequencing of the mixture of barcoded DNA fragments.

In some embodiments, the DNA is genomic DNA or mitochondrial DNA. In some embodiments, the DNA is genomic DNA and the method further comprises depleting nucleosomal or histone proteins from the lysed cells before the contacting. In some embodiments, the nucleic acids are RNA and the method comprises converting the RNA into DNA with a reverse transcriptase.

In some embodiments, the method further comprises: after the contacting and before the partitioning, contacting the DNA fragments in the hydrogel beads or SPCs with (i) a tet methylcytosine dioxygenase 2 (TET2) that catalyzes conversion of 5-methylcytosine to 5-hydroxymethylcytosine (5hmC) and then 5-carboxylcytosine (5caC) in the DNA fragments or (ii) a beta-glucosyltransferase that catalyzes conversion of 5-methylcytosine to 5-hydroxymethylcytosine (5-hmC) residues and then beta-glucosyl-5-hydroxymethylcytosine (5gmC) in the DNA fragments; and after the forming, contacting the barcoded DNA fragments with a DNA cytidine deaminase that deaminates cytosine but not 5caC or 5gmC. In some embodiments, the DNA cytidine deaminase is APOBEC3A.

In some embodiments, a majority of microwells containing a cell reaction hydrogel bead or SPC contain only one cell reaction hydrogel bead or SPC.

In some embodiments, the cell reaction hydrogel beads, the barcoding hydrogel beads, or both, comprise cross-linked alginate. In some embodiments, the dissolving comprises contacting the cross-linked alginate with a calcium chelator. In some embodiments, the calcium chelator is EDTA or sodium citrate.

In some embodiments, the depleting of nucleosomal proteins from the lysed cells comprises contacting genomic DNA from the lysed cells with a protease, a detergent, or both a protease and a detergent.

In some embodiments, the method further comprises, between the partitioning and the dissolving, sealing the microwells from each other with a water-impermeable barrier. In some embodiments, the sealing comprises applying a layer of oil to cover the microwells.

In some embodiments, the cells are mammalian cells. In some embodiments, the cells are bacterial or plant cells.

In some embodiments, the performing nucleotide sequencing of the mixture comprises nucleotide sequencing of the first and second barcoded strand of barcoded genomic double-stranded DNA fragments.

In some embodiments, the first strand of the adaptor oligonucleotides comprise 5′-3′: a spacer sequence, one or more uracil or modified bases or carbon spacer and a transposase binding (ME) sequence and further comprising amplifying the first and/or second barcoded strand of barcoded genomic double-stranded DNA fragments with a polymerase that stops primer extension at the one or more uracil or modified bases or carbon spacer to form a truncated amplicon.

In some embodiments, the method further comprises amplifying the first and/or second barcoded strand of barcoded genomic double-stranded DNA fragments or the truncated amplicon with a first primer that anneals to the ME sequence. In some embodiments, the amplifying further comprises amplifying the first and/or second barcoded strand of barcoded genomic double-stranded DNA fragments or the truncated amplicon with a second primer that anneals to the first strand of the adaptor oligonucleotide such that a resulting amplification product comprises the barcode sequence.

In some embodiments, the method further comprises, before the lysing, contacting the cells with one or more different antibodies, wherein each antibody is linked to an antibody oligonucleotide comprising an antibody barcode sequence specific for the antibody and a 5′ alkyne moiety, and wherein the linking further comprises linking the 5′ alkyne moiety on the antibody oligonucleotide to the 3′ azide moiety of the barcoding oligonucleotides via click chemistry to form a DNA molecule comprising the antibody-barcode and barcode sequence that identifies the barcoding hydrogel bead; and nucleotide sequencing of DNA molecules comprising the antibody-barcode and barcode sequence that identifies the barcoding hydrogel bead. In some embodiments, the antibodies bind to surface antigens on the cells. In some embodiments, the cells are permeabilized and the antibodies bind to antigens in the cells. In some embodiments, the contacting of the cells with the one or more different antibodies occurs before the forming. In some embodiments, the contacting of the cells with the one or more different antibodies occurs after the forming.

In some embodiments, the method comprises providing a plurality of microwells containing alginic acid;
introducing into the microwells (i) single cells and (ii) barcoding hydrogel beads linked to barcoding oligonucleotides comprising (i) a barcode sequence that identifies the barcoding hydrogel bead and (ii) a 3′ azide moiety;
inducing gelation of the alginate to form an alginate matrix surrounding the cells in the microwells;
diffusing into the microwells reagents that lyse the cells, thereby releasing nucleic acids from the cells, wherein the nucleic acids are DNA from the cell or RNA, and optionally converting the RNA into DNA with a reverse transcriptase;
contacting the DNA of the lysed cells with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked genomic DNA fragment having the 5′ alkyne moieties;
dissolving the alginate matrix and the barcoding hydrogel beads in the microwells;
linking the 5′ alkyne moieties of the adaptor-linked DNA fragments to the 3′ azide moiety of the barcoding oligonucleotides via click chemistry to form a first and second barcoded strand of barcoded genomic double-stranded DNA fragments;
recovering barcoded DNA fragments from the microwells and forming a mixture of barcoded DNA fragments from different microwells; and
performing nucleotide sequencing of the mixture of barcoded DNA fragments.

In some embodiments, the DNA is genomic DNA or mitochondrial DNA. In some embodiments, the DNA is genomic DNA and the method further comprises depleting nucleosomal or histone proteins from the lysed cells before the contacting.

In some embodiments, the nucleic acids are RNA and the method comprises converting the RNA into DNA with a reverse transcriptase.

In some embodiments, the method further comprises after the contacting and before the linking, contacting the genomic DNA fragments with (i) a tet methylcytosine dioxygenase 2 (TET2) that catalyzes conversion of 5-methylcytosine to 5-hydroxymethylcytosine (5hmC) and then 5-carboxylcytosine (5caC) in the DNA fragments or (ii) a beta-glucosyltransferase that catalyzes conversion of 5-methylcytosine to 5-hydroxymethylcytosine (5-hmC) residues and then beta-glucosyl-5-hydroxymethylcytosine (5gmC) in the DNA fragments; and after the forming, contacting the barcoded genomic DNA fragments with a DNA cytidine deaminase that deaminates cytosine but not 5caC or 5gmC. In some embodiments, the DNA cytidine deaminase is APOBEC3A. In some embodiments, a majority of microwells containing a cell contains only one cell. In some embodiments, the dissolving comprises contacting the alginate matrix and barcoding hydrogel beads with a calcium chelator. In some embodiments, the calcium chelator is EDTA or sodium citrate.

In some embodiments, the depleting of nucleosomal proteins from the lysed cells comprises contacting DNA from the lysed cells with a protease, a detergent, or both a protease and a detergent.

In some embodiments, the method further comprises, before the gelation, sealing the microwells from each other with a water-impermeable barrier. In some embodiments, the sealing comprises applying a layer of oil to cover the microwells.

In some embodiments, the method further comprises, before or during the dissolving, sealing the microwells from each other with a water-impermeable barrier. In some embodiments, the sealing comprises applying a layer of oil to cover the microwells.

In some embodiments, the cells are mammalian cells. In some embodiments, the cells are bacterial or plant cells.

In some embodiments, the performing nucleotide sequencing of the mixture comprises nucleotide sequencing of the first and second barcoded strand of barcoded double-stranded DNA fragments.

In some embodiments, the first strand of the adaptor oligonucleotides comprise 5′-3′: a spacer sequence, one or more uracil or modified bases or a carbon spacer and a transposase binding (ME) sequence and further comprising amplifying the first and/or second barcoded strand of barcoded genomic double-stranded DNA fragments with a polymerase that stops primer extension at the one or more uracil or modified bases or carbon spacer to form a truncated amplicon.

In some embodiments, the method further comprises amplifying the first and/or second barcoded strand of barcoded double-stranded DNA fragments or the truncated amplicon with a first primer that anneals to the ME sequence. In some embodiments, the amplifying further comprises amplifying the first and/or second barcoded strand of barcoded genomic double-stranded DNA fragments or the truncated amplicon with a second primer that anneals to the first strand of the adaptor oligonucleotide such that a resulting amplification product comprises the barcode sequence.

Also provided is a plurality of microwells. In some embodiments, the microwells contain:
(i) a cell reaction hydrogel bead or SPC comprising DNA fragments and
(ii) a barcoding hydrogel bead linked to barcoding oligonucleotides comprising (a) a barcode sequence that identifies the barcoding hydrogel bead and (b) a 3′ azide moiety.

In some embodiments, the genomic fragments were formed by contacting DNA of lysed cells in cell reaction hydrogel beads with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked DNA fragment having the 5′ alkyne moieties.

Also provided is a mixture comprising a plurality of first and second barcoded strands of barcoded double-stranded DNA fragments, wherein the first and second barcodes strands comprise an identical barcode sequence.

Also provided is a method of barcoding DNA. In some embodiments, the method comprises
contacting DNA with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked DNA fragment having the 5′ alkyne moieties;
mixing the DNA fragments, optionally from a single cell, with a barcoding bead linked to barcoding oligonucleotides comprising (i) a barcode sequence that identifies the barcoding bead and (ii) a 3′ azide moiety; and
linking the 5′ alkyne moieties of the adaptor-linked DNA fragments to the 3′ azide moiety of the barcoding oligonucleotides via click chemistry to form a first and second barcoded strand of barcoded double-stranded DNA fragments, thereby barcoding the DNA.

In some embodiments, the method further comprises, before the contacting, introducing cells into partitions and lysing the cells, and wherein the contacting occurs in the partitions. In some embodiments, the partitions are hydrogel beads, droplets, SPCs or microwells.

In some embodiments, the method further comprises, after the introducing and before the contacting, converting the RNA into DNA with a reverse transcriptase, and wherein the DNA is cDNA.

In some embodiments, the method further comprises performing nucleotide sequencing of polynucleotides comprising the barcoded DNA fragments.

Also provided is a method of nucleotide sequencing, the method comprising
forming cell reaction hydrogel beads or SPCs comprising single cells;
lysing the cells in the cell reaction hydrogel beads or SPCs such that at least a majority of nucleic acids of the cells is retained in the cell reaction hydrogel beads or SPCs, wherein the nucleic acids are DNA from the cell or RNA, and optionally converting the RNA into DNA with a reverse transcriptase;
contacting the DNA in the cell reaction hydrogel beads or SPCs with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, thereby forming adaptor-linked DNA fragments;
gap-filling the adaptor-linked DNA fragments to form gap-filled adaptor-linked DNA fragments;
partitioning in microwells the cell reaction hydrogel beads or SPCs comprising the gap-filled adaptor-linked DNA fragments with a barcoding hydrogel bead linked to barcoding oligonucleotides comprising (i) a barcode sequence that identifies the barcoding hydrogel bead and (ii) a 3′ capture sequence that anneals to a 3′ end of the gap-filled adaptor-linked DNA fragments, thereby forming microwells containing one of the cell reaction hydrogel beads or SPCs and one of the barcoding hydrogel beads;
dissolving the cell reaction hydrogel beads or SPCs and barcoding hydrogel beads in the microwells;
after the dissolving, extending the 3′ capture sequence annealed to the 3′ ends of the gap-filled adaptor-linked DNA fragments using the gap-filled adaptor-linked DNA fragments as a template to form barcoded DNA fragments,
recovering barcoded DNA fragments from the microwells and forming a mixture of barcoded DNA fragments from different microwells; and
performing nucleotide sequencing of the mixture of barcoded DNA fragments.

In some embodiments, the DNA is genomic DNA or mitochondrial DNA. In some embodiments, the DNA is genomic DNA and the method further comprises depleting nucleosomal proteins from the lysed cells before the contacting.

In some embodiments, the nucleic acids are RNA and the method comprises converting the RNA into DNA with a reverse transcriptase.

In some embodiments, the method further comprises, after the contacting and before the partitioning, contacting the DNA fragments in the hydrogel beads or SPCs with a tet methylcytosine dioxygenase 2 (TET2) enzyme, and optionally further comprising beta-glucosyltransferase, that catalyzes conversion of 5-methylcytosine to 5-hydroxymethylcytosine in the DNA fragments; and after the forming, contacting the barcoded DNA fragments with a DNA cytidine deaminase. In some embodiments, the DNA cytidine deaminase is APOBEC3A.

In some embodiments, a majority of microwells containing a cell reaction hydrogel bead or SPC contains only one cell reaction hydrogel bead or SPC.

In some embodiments, the cell reaction hydrogel beads, the barcoding hydrogel beads, or both, comprise cross-linked alginate. In some embodiments, the dissolving comprises contacting the cross-linked alginate with a calcium chelator. In some embodiments, the calcium chelator is EDTA or sodium citrate.

In some embodiments, the depleting of nucleosomal proteins from the lysed cells comprises contacting genomic DNA from the lysed cells with a protease, a detergent, or both a protease and a detergent.

In some embodiments, between the partitioning and the dissolving, sealing the microwells from each other with a water-impermeable barrier. In some embodiments, the sealing comprises applying a layer of oil to cover the microwells.

In some embodiments, the cells are mammalian cells. In some embodiments, the cells are bacterial or plant cells.

### BRIEF DESCRIPTION OF THE DRAWINGS

FIG. 1A depicts (1) forming hydrogel beads containing single cells, (2) cell-lysis or permeabilization of cells, and (3) depletion nucleosomes from chromosomal DNA from the cells.

FIG. 1B depicts Optional conversion of methylated bases using various enzymes including for example tet and APOBEC.

FIG. 1C depicts (4) tagmentation (i.e., causing DNA fragmentation with a transposase that introduced oligonucleotides to the ends of the fragments), (5) an option of treating the DNA in the hydrogel beads with TET2, causing one or more of the changes indicated in FIG. 1B, (6) dissolving the cell reaction hydrogel beads (cell bead) and barcoding hydrogel bead (barcode bead) and linking the barcoding oligonucleotides to the DNA fragments from the cells by click chemistry, (7) combining the contents of the wells into a bulk solution, and (8) optionally treating the bulk DNA solution (if the DNA was previously treated with TET2) with APOBEC3A.

FIG. 2A depicts an alternative to that shown in FIG. TA-C. In FIG. 2A wells are provided containing alginic acid, which can later be gelated upon addition of calcium.

FIG. 2B depicts introduction of single cells as well as alginate barcoding hydrogel beads into the wells. The conditions can be selected to achieve a single cell and single alginate barcoding hydrogel bead into wells. The alginic acid in the wells can subsequently be gelled, for example by adding calcium to the wells, thereby forming alginate matrices (see, g., FIG. 5). This can be achieved under a layer of oil, preventing significant exchange between the wells prior to formation of the alginate matrix.

FIG. 2C depicts diffusion of reagents into the alginate matrices in the wells. Exemplary reagents can include, for example, buffers that result in cell lysis and/or nucleosome or histone depletion of DNA from the cells. in the alginate matrices. Subsequently (bottom of figure), the alginate matrices can be dissolved, for example by contacting the matrices with a calcium chelator and then barcoding of DNA fragments with barcoding oligonucleotides can be initiated by click chemistry.

FIG. 3 depicts linkage of DNA (e.g., a DNA fragment and barcoding oligonucleotide) with click chemistry. The resulting product can in some embodiments be amplified via primer extension/PCR such that the resulting products have standard phosphate linkage.

FIG. 4A depicts an optional workflow in which cell reaction hydrogel beads (gDNA bead) and barcoding hydrogel beads (CBC bead) are co-partitioned into wells. The bottom portion of the figure depicts a single exemplary cell reaction hydrogel beads (gDNA bead) in which tagmentation occurs. Depicted is a single DNA fragment (or many) resulting from the tagmentation in which the 5′ ends of each strand of the fragment is linked to 3′ ends of the first strand of the adaptor oligonucleotides delivered by the tagmentase (transposase). “ME” refers to the mosaic end sequence (e.g., SEQ ID NO:1) recognized by the tagmentase. The oligonucleotide further comprises an optional spacer sequence, wherein optionally the spacer and ME sequences are separated by a nucleotide or nucleotides (represented as a diamond), which certain polymerases cannot process through. Finally, the 5′ end of the oligonucleotide transferred to the DNA fragment is an alkyne that can be used for click chemistry linkage. Both strands of the DNA fragment in some embodiments receive an identical copy of the adapter oligonucleotide. The figure also shows a hydrogel (alginate) barcoding bead with barcoding oligonucleotides having a 3′ azide that are linked to the tagmented DNA fragment via click chemistry.

FIG. 4C depicts at its top the product of the FIG. 4B reaction. As shown in the middle portion of FIG. 4C different microwells will contain the same products, albeit with different barcoding oligonucleotides linked via click chemistry. At the bottom of FIG. 4B the barcoded DNA fragments are combined into a mixture.

FIG. 4D depicts purification of barcoded DNA fragments in bulk. As shown in this figure, in some embodiments the nucleotide or nucleotides (represented as a diamond) that certain polymerases cannot process through can be a plurality of uracils. For purification, in some embodiments, DNA-binding magnetic beads are added to the microwells, which contains barcoded DNAs. After incubation, DNA-bound magnetic beads are removed by a magnet and then washed to remove carryover chemicals. After washing, purified DNAs that are bound to the magnetic beads are eluted for subsequent library preparation steps.

FIG. 4E depicts a gap-filling reaction using a polymerase sensitive to the nucleotide or nucleotides represented as a diamond (e.g., poly uracil). Because the polymerase is sensitive to the nucleotides, extension terminate at their position, resulting in an amplicon lacking the subsequent 5′ portion. Optionally, NaOH denaturation, neutralization, and APOEC3A treatment can occur.

FIG. 4F depicts optional amplification schemes in which a target-specific primer or an ME-specific primer (for whole genome pre-amplification) is used to amplify the resulting products that can subsequently be submitted to next-generation sequencing (NGS). In some embodiments, a ME adapter primer and a barcode adapter primer is used, for example for whole genome amplification and sequencing. In some embodiments, a gene-specific adapter primer and a barcode adapter primer is used, for example, for targeted and sense/anti-sense strand amplification and sequencing. In some embodiments, a gene-specific forward and a reverse adapter-primer and a barcode adapter is used, for example, for targeted non-strand specific amplification and sequencing.

FIG. 5 depicts a mechanism of reversible alginate matrix formation. In some embodiments, the wells contain alginic acid and Ca-EDTA, while the oil that seals the well contains acetic acid. As a result, after well sealing, the lower pH environment breaks the bonding between Ca and EDTA. Free Ca will therefore cross-link the alginic acid ionically to form a gel in well. See also FIG. 2A.

FIG. 6 depicts an exemplary alkyne having a 5′ hexynyl.

FIG. 7 depicts an exemplary azide-ddNTP.

FIG. 8 depicts a microscopic view of single-cell encapsulated alginate hydrogel bead after cell-lysis/nucleosome depletion.

FIGS. 9A-9D depict results from BioAnalyzer profiling of single-cell encapsulated alginate hydrogel bead after cell-lysis/nucleosome depletion and tagmentation processes.

### DEFINITIONS

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Amplification reaction mixtures may also further include stabilizers and other additives to optimize efficiency and specificity. Depending upon the context, the mixture can be either a complete or incomplete amplification reaction mixture

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

“Primer extension” refers to any method in which a primer is extended in a template-specific manner. Examples of primer extension include, for example, methods in which a primer hybridizes to a template nucleic acid and a polymerase extends the primer in a template-specific manner. In some embodiments, the template is DNA and the polymerase is a DNA polymerase. In some embodiments, the template is RNA and the polymerase is a reverse-transcriptase. Primer extension can also include, for example, template switching (see, e.g., Zhu Y Y, Machleder E M, et al. (2001) Biotechniques, 30(4):892-897; Ramskold D, Luo S, et al. (2012) Nat Biotechnol, 30(8):777-78, and nick polymerization (also referred to as nick translation), the latter involving nicking one strand of a nucleic acid duplex and using the nicked strand as a primer that is extended using the other strand as a template (see, e.g., Leonard G. Davis Ph.D., et al, in Basic Methods in Molecular Biology, 1986).

A nucleic acid, or a portion thereof, “hybridizes” or “anneals” to another nucleic acid under conditions such that non-specific hybridization is minimal at a defined temperature in a physiological buffer (e.g., pH 6-9, 25-150 mM chloride salt or in a PCR reaction mixture). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C. In some embodiments, the defined temperature at which specific hybridization occurs is 37, 40, 42, 45, 50, 55, 60, 65, 70, 75, or 80° C.

A “template” refers to a polynucleotide sequence that comprises the polynucleotide to be amplified, flanked by or a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

As used herein a “barcode” is a short nucleotide sequence (e.g., at least about 4, 6, 8, 10, 12, 14, 16, 18, 20 or more nucleotides long) that identifies a molecule to which it is conjugated. Barcodes can be used, e.g., to identify molecules in a partition. Such a partition-specific barcode should be unique for that partition as compared to barcodes present in other partitions. For example, partitions containing target RNA from single-cells can subject to reverse transcription conditions using primers that contain a different partition-specific barcode sequence in each partition, thus incorporating a copy of a unique “cellular barcode” into the reverse transcribed nucleic acids of each partition. Thus, nucleic acid from each cell can be distinguished from nucleic acid of other cells due to the unique “cellular barcode.” In some cases, the cellular barcode is provided by a “bead barcode” that is present on oligonucleotides conjugated to a bead, wherein the bead barcode is shared by (e.g., identical or substantially identical amongst) all, or substantially all, of the oligonucleotides conjugated to that bead but is different from most or substantially all oligonucleotides conjugated to other beads. Thus, cellular and bead barcodes can be present in a partition, attached to a bead, or bound to cellular nucleic acid as multiple copies of the same barcode sequence. Cellular or bead barcodes of the same sequence can be identified as deriving from the same cell, partition, or bead. Such partition-specific, cellular, or bead barcodes can be generated using a variety of methods, which methods result in the barcode conjugated to or incorporated into a solid or hydrogel support (e.g., a solid bead or particle or hydrogel bead or particle). In some cases, the partition-specific, cellular, or bead barcode is generated using a split and mix (also referred to as split and pool) synthetic scheme as described herein. A partition-specific barcode can be a cellular barcode and/or a bead barcode (for example when associated with a cell or partition or both). Similarly, a cellular barcode can be a partition specific barcode (when provided in a partition) and/or a bead barcode (when delivered by a bead). Additionally, a bead barcode can be a cellular barcode and/or a partition-specific barcode.

In other cases, barcodes uniquely identify the molecule to which it is conjugated and are referred to as a unique molecular identifier (UMI). The number of nucleotides of the UMI, which can be continuous, or discontinuous, will depend on the number of UMI sequences required. In some embodiments, the number of UMIs available are many times (e.g., 2×, 10×, 100×, etc) higher than possible conjugation partners, thereby reducing the chance of rare duplicates being linked to different molecules. In some embodiments, pools of different UMIs are present in a partition and the composition of the pool acts as an identifiers for the partition, with some UMIs being in common with some other partitions but the total pool of UMIs being unique or substantially unique between partitions. UMI sequences can be generated for example as random sequences of a set length, and in some embodiments is identified by a flanking known sequence.

The length of the barcode sequence determines how many unique samples can be differentiated. For example, a 1 nucleotide barcode can differentiate 4, or fewer, different samples or molecules; a 4-nucleotide barcode can differentiate 4⁴ or 256 samples or less; a 6 nucleotide barcode can differentiate 4096 different samples or less; and an 8 nucleotide barcode can index 65,536 different samples or less. Additionally, barcodes can be attached to both strands either through barcoded primers for both first and second strand synthesis, through ligation, or in a tagmentation reaction.

Barcodes are typically synthesized and/or polymerized (e.g., amplified) using processes that are inherently inexact. Thus, barcodes that are meant to be uniform (e.g., a cellular, particle, or partition-specific barcode shared amongst all barcoded nucleic acid of a single partition, cell, or bead) can contain various N−1 deletions or other mutations from the canonical barcode sequence. Thus, barcodes that are referred to as “identical” or “substantially identical” copies refer to barcodes that differ due to one or more errors in, e.g., synthesis, polymerization, or purification errors, and thus contain various N−1 deletions or other mutations from the canonical barcode sequence. Moreover, the random conjugation of barcode nucleotides during synthesis using e.g., a split and pool approach and/or an equal mixture of nucleotide precursor molecules as described herein, can lead to low probability events in which a barcode is not absolutely unique (e.g., different from all other barcodes of a population or different from barcodes of a different partition, cell, or bead). However, such minor variations from theoretically ideal barcodes do not interfere with the high-throughput sequencing analysis methods, compositions, and kits described herein. Therefore, as used herein, the term “unique” in the context of a particle, cellular, partition-specific, or molecular barcode encompasses various inadvertent N−1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences). For example, 10,000 cells can be analyzed using a cellular barcode having 9 barcode nucleotides, representing 262,144 possible barcode sequences. The use of barcode technology is well known in the art, see for example Katsuyuki Shiroguchi, et al. Proc Natl Acad Sci USA., 2012 Jan. 24; 109(4):1347-52; and Smith, A M et al., Nucleic Acids Research Can 11, (2010). Further methods and compositions for using barcode technology include those described in U.S. 2016/0060621.

A “transposase” or “tagmentase” means an enzyme that is capable of forming a functional complex with a transposon end-containing composition and catalyzing insertion or transposition of the transposon end-containing composition into the double-stranded target DNA with which it is incubated in an in vitro transposition reaction.

The term “transposon end” means a double-stranded DNA that exhibits the nucleotide sequences (the “transposon end sequences”) that are necessary to form the complex with the transposase that is functional in an in vitro transposition reaction. A transposon end forms a “complex” or a “synaptic complex” or a “transposome complex” or a “transposome composition” with a transposase or integrase that recognizes and binds to the transposon end, and which complex is capable of inserting or transposing the transposon end into target DNA with which it is incubated in an in vitro transposition reaction. A transposon end exhibits two complementary sequences consisting of a “transferred transposon end sequence” or “transferred strand” and a “non-transferred transposon end sequence,” or “non transferred strand” For example, one transposon end that forms a complex with a hyperactive Tn5 transposase (e.g., EZ-Tn5™ Transposase, EPICENTRE Biotechnologies, Madison, Wis., USA) that is active in an in vitro transposition reaction comprises a transferred strand that exhibits a “transferred transposon end sequence” as follows:

(SEQ ID NO: 3)
5′ AGATGTGTATAAGAGACAG 3′,

and a non-transferred strand that exhibits a “non-transferred transposon end sequence” as follows:

(SEQ ID NO: 6)
5′ CTGTCTCTTATACACATCT 3′.

The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

The term “solid support” refers to the surface of a bead, microtiter well or other surface that is useful for attaching a nucleic acid, such as an oligonucleotide or polynucleotide. The surface of the solid support can be treated to facilitate attachment of a nucleic acid, such as a single stranded nucleic acid.

The term “bead” refers to any solid support that can be in a partition, e.g., a small particle or other solid support. In some embodiments, the beads comprise an alginate matrix, i.e., calcium alginate. In some embodiments, the beads comprise polyacrylamide. For example, in some embodiments, the beads incorporate barcode oligonucleotides into the gel matrix through an acrydite chemical modification attached to each oligonucleotide. Exemplary beads can include hydrogel beads. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240.

It will be understood that any range of numerical values disclosed herein can include the endpoints of the range, and any values or subranges in between the endpoints. For example, the range 1 to 10 includes the endpoints 1 and 10, and any value between 1 and 10. The values typically include one significant digit.

The term “sample” refers to a biological composition, such as a cell, comprising a target nucleic acid.

The term “about” refers to the usual error range for the respective value that is known by a person of ordinary skill in the art for this technical field, for example, a range of +10%, +5%, or +1% can encompass the recited value, even if the recited value is not modified by the term “about.”

All ranges described herein can include the end point values of the range, and any sub-range of values included between the endpoints of the range, where the values include the first significant digit. For example, a range of 1 to 10 includes a range from 2 to 9, 3 to 8, 4 to 7, 5 to 6, 1 to 5, 2 to 5, 2 to 10, 3 to 10, and so on.

### DETAILED DESCRIPTION OF THE INVENTION

### Introduction

Methods and compositions for barcoding nucleic acids from single cells and nucleotide sequencing are provided. In some aspects, an adapter oligonucleotide (introduced by a transposase) at the end of DNA fragments is linked to a partition-specific barcoding oligonucleotide using click chemistry. In some aspects, adapters are introduced on both ends of a DNA fragment due to the nature of forming DNA fragments with a transposase, with each strand receiving an adaptor oligonucleotide with a 5′ alkyne. Subsequent contact of the DNA fragments from a single cell with a partition-specific barcoding oligonucleotide having a 3′ azide allows for linkage of both strands of the adaptor-labeled DNA fragments to a copy of the partition-specific barcode oligonucleotide.

Also provided are methods of associating DNA from single cells with a partition-specific barcoding oligonucleotide. In these aspects, single cells can be provided in hydrogel beads or semi-permeable capsules (SPCs). Lysis of the cells within the hydrogel beads or SPCs retain the macromolecules such as nucleic acids in the beads. The nucleic acids in the beads can be molecularly manipulated by diffusing in enzymes and reagents, for example allowing for tagmentation (fragmentation via a transposase) of DNA in the beads, resulting in adaptor-labeled DNA fragments. The DNA can be DNA originating in the cells (e.g., genomic DNA or mitochondrial DNA) or can be cDNA generated by introducing reverse transcriptase and regents allowing for generation of cDNAs from RNA in the cells or in other embodiments, DNA tags introduced by binding agents such as antibodies. The hydrogel beads containing the single lysed cells and having tagmented DNA can be introduced into microwells or other partitions along with a barcoding hydrogel bead linked to barcoding oligonucleotides comprising (i) a barcode sequence that identifies the barcoding hydrogel bead and (ii) a capture sequence allowing for annealing to the tagmented DNA fragments of requisite click chemistry at the 3′ end. In other aspects, single cells can be delivered directly to the microwells or other partitions along with the barcoding hydrogel bead and the microwell or other partition can subsequently be encompassed in an alginate matrix, allowing for the same molecular manipulation discussed above within beads. In either embodiment, the barcoding oligonucleotides can be linked via primer extension or ligation or click chemistry to the DNA fragments in the microwells. Subsequently, the barcoded DNA fragments can be combined in bulk (i.e., different microwell contents can be mixed), optionally purified, and the resulting mixture can be prepared for nucleotide sequencing. In view of the barcoding, nucleotide sequencing reads can be used to identify sequencing reads for different cells. The methods described in this paragraph can employ click chemistry as discussed elsewhere herein to link the transposase oligonucleotides to the barcoding oligonucleotides, or not, in which case primer extension to ligation can be used to link the transposase oligonucleotides to the barcoding oligonucleotide sequences.

In a first aspect, methods and compositions for using click chemistry to link transposase-treated DNA fragments to barcoding oligonucleotides are provided. Click chemistry involves an azide alkyne Huisgen cycloaddition reaction, wherein in the embodiments described herein, a first oligonucleotide having an end alkyne moiety is reacted with a second oligonucleotide having an end azide moiety and promoting or catalyzing a reaction between the moieties to covalently-link the two oligonucleotides. In some embodiments, the azide-alkyne Huisgen cycloaddition is a 1,3-dipolar cycloaddition between an azide and a terminal or internal alkyne to give a 1,2,3-triazole. The reaction can in some embodiments be catalyzed by copper (Copper(I)-catalyzed azide-alkyne cycloaddition (CuAAC)) or promoted by a strained difluorooctyne (DIFO) (Strain-promoted azide-alkyne cycloaddition (SPAAC)) for example. See, e.g., Chemical Reviews: Click Chemistry, Jun. 23, 2021, Volume 121, Issue 12, Pages 6697-7248, including for example, Fantoni et al., “A Hitchhiker's Guide to Click-Chemistry with Nucleic Acids” Chem. Rev. 2021, 121, 7122-7154. Thus in some embodiments, click chemistry ligation chemically attaches a 5′ alkyne moiety of tagmented DNA with a 3′ Azido moiety at the terminus of a barcode oligonucleotide. The click ligation can be proceeded in some embodiments, by an incubation at 45 degrees C., for example, for 2 hours in the presence of Vitamin C, Cu(II)-TBTA, MgSO4, THPTA, and DMSO. In the aspects described herein, the resulting product of click chemistry ligation should still be available as a template of DNA polymerization.

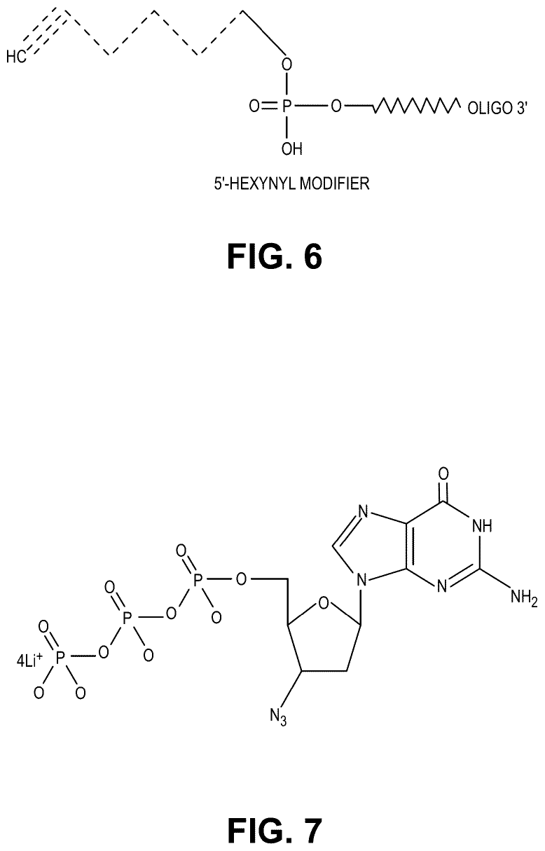

Click chemistry can be used to link the fragmented DNA to a barcoding oligonucleotide. In these embodiments, the 5′ end of the oligonucleotide transferred strand (also referred to herein as the “first strand”) by the transposase to the DNA (which is fragmented in the process) comprises one of the reactive moieties for click chemistry (i.e., an azide or an alkyne moiety) and a '3 end of the barcoding oligonucleotide has the corresponding click chemistry moiety (i.e., an alkyne or an azide, respectively). In some embodiments, the 5′ end of the oligonucleotide transferred strand comprises an alkyne moiety and the '3 end of the barcoding oligonucleotide comprises an azide moiety. In some embodiments, an alkyne adapter (e.g., subsequently loaded onto a transposase) is chemically synthesized to add 5′Hexynyl to the 5′ phosphate of oligonucleotide. See, e.g., FIG. 6. In some embodiments, an azide is added to a'ssDNA oligonucleotide by terminal transferase TdT through an azide-ddNTP, which as N3 (azide) placed at the original 3′OH position. See, e.g., FIG. 7.

As noted above, the 5′ end of the oligonucleotide transferred strand (also referred to herein as the “first strand”) by the transposase can comprise an alkyne moiety. The action of the transposase is sometimes referred to as “tagmentation” and can involve introduction of different adapter sequences on different sides of a DNA breakage point caused by the transposase or the adapter sequences added can be identical. In either case, the one or two adapter sequences are common adapter sequences in that the adapter sequences are the same across a diversity of DNA fragments. Homoadapter-loaded tagmentases are tagmentases that contain adapters of only one sequence, which adapter is added to both ends of a tagmentase-induced breakpoint in the genomic DNA. Heteroadapter-loaded tagmentases are tagmentases that contain two different adapters, such that a different adapter sequence is added to the two DNA ends created by a tagmentase-induced breakpoint in the DNA. Adapter loaded tagmentases are further described, e.g., in U.S. Patent Publication Nos: 2010/0120098; 2012/0301925; and 2015/0291942 and U.S. Pat. Nos. 5,965,443; 6,437,109; 7,083,980; 9,005,935; and 9,238,671, the contents of each of which are hereby incorporated by reference in the entirety for all purposes. Tagmentation of RNA/DNA hybrids is described in, e.g., Bo LuLiting et al., eLife 9:e54919 (2020). As described herein, the strand of nucleic acid adapter transferred will comprise a 5′ alkyne moiety.

A tagmentase is an enzyme that is capable of forming a functional complex with a transposon end-containing composition and catalyzing insertion or transposition of the transposon end-containing composition into the double-stranded target DNA with which it is incubated in an in vitro transposition reaction. Exemplary transposases include but are not limited to modified Tn5 transposases that are hyperactive compared to wildtype Tn5, for example can have one or more mutations selected from E54K, M56A, or L372P. Wild-type Tn5 transposon is a composite transposon in which two near-identical insertion sequences (IS50L and IS50R) are flanking three antibiotic resistance genes (Reznikoff WS. Annu Rev Genet 42: 269-286 (2008)). Each IS50 contains two inverted 19-bp end sequences (ESs), an outside end (OE) and an inside end (IE). However, wild-type ESs have a relatively low activity and were replaced in vitro by hyperactive mosaic end (ME) sequences. A complex of the transposase with the 19-bp ME is thus all that is necessary for transposition to occur, provided that the intervening DNA is long enough to bring two of these sequences close together to form an active Tn5 transposase homodimer (Reznikoff WS., et al. Mol Microbiol 47: 1199-1206 (2003)). Transposition is a very infrequent event in vivo, and hyperactive mutants were historically derived by introducing three missense mutations in the 476 residues of the Tn5 protein (E54K, M56A, L372P), which is encoded by IS50R (Goryshin I Y, Reznikoff WS. 1998. J Biol Chem 273: 7367-7374 (1998)). Transposition works through a “cut-and-paste” mechanism, where the Tn5 excises itself from the donor DNA and inserts into a target sequence, creating a 9-bp duplication of the target (Schaller H. Cold Spring Harb Symp Quant Biol 43: 401-408 (1979); Reznikoff W S., Annu Rev Genet 42: 269-286 (2008)). In current commercial solutions (Nextera™ DNA kits, Illumina), free synthetic ME adapters are end-joined to the 5′-end of the target DNA by the transposase (tagmentase).

An “adaptor oligonucleotide” refers to an oligonucleotide that carries a universal sequence, wherein the universal sequences are common to end sequences between different fragments to which the oligonucleotide adaptor is attached and permit their use as PCR handle sequences, allowing one pair of universal primers to amplify different fragments that have the universal sequences. In some embodiments, the adapter(s) is at least 19 nucleotides in length, e.g., 19-100 nucleotides. In some embodiments, the adapters are double stranded with a 5′ end overhang, wherein the 5′ overhand sequence is different between heteroadapters, while the double stranded portion (typically 19 bp) is the same. In some embodiments, an adapter comprises TCGTCGGCAGCGTC (SEQ ID NO:1) or GTCTCGTGGGCTCGG (SEQ ID NO:2). In some embodiments involving the heteroadapter-loaded tagmentase, the tagmentase is loaded with a first adapter comprising TCGTCGGCAGCGTC (SEQ ID NO:1) and a second adapter comprising GTCTCGTGGGCTCGG (SEQ ID NO:2). In some embodiments, the adapter comprises AGATGTGTATAAGAGACAG (SEQ ID NO:3) and the complement thereof (this is the mosaic end and this is the only specifically required cis active sequence for Tn5 transposition). In some embodiments, the adapter comprises TCGTCGGCAGCGTCAGATGTGTATAAGAGACAG (SEQ ID NO:4) with the complement for AGATGTGTATAAGAGACAG (SEQ ID NO:3) or GTCTCGTGGGCTCGGAGATGTGTATAAGAGACAG (SEQ ID NO: 5) with the complement for AGATGTGTATAAGAGACAG (SEQ ID NO:3). In some embodiments involving the heteroadapter-loaded tagmentase, the tagmentase is loaded with a first adapter comprising TCGTCGGCAGCGTCAGATGTGTATAAGAGACAG (SEQ ID NO:4) with the complement for AGATGTGTATAAGAGACAG (SEQ ID NO:3) (which can have a 5′phosphate to allow for loading on a transposase) and GTCTCGTGGGCTCGGAGATGTGTATAAGAGACAG (SEQ ID NO:5) with the complement for AGATGTGTATAAGAGACAG (SEQ ID NO:3). Any of SEQ ID Nos 4 or 5 oligonucleotides can have a 5′ phosphate modified with a 5′ hexynyl group, providing the alkyne moiety.

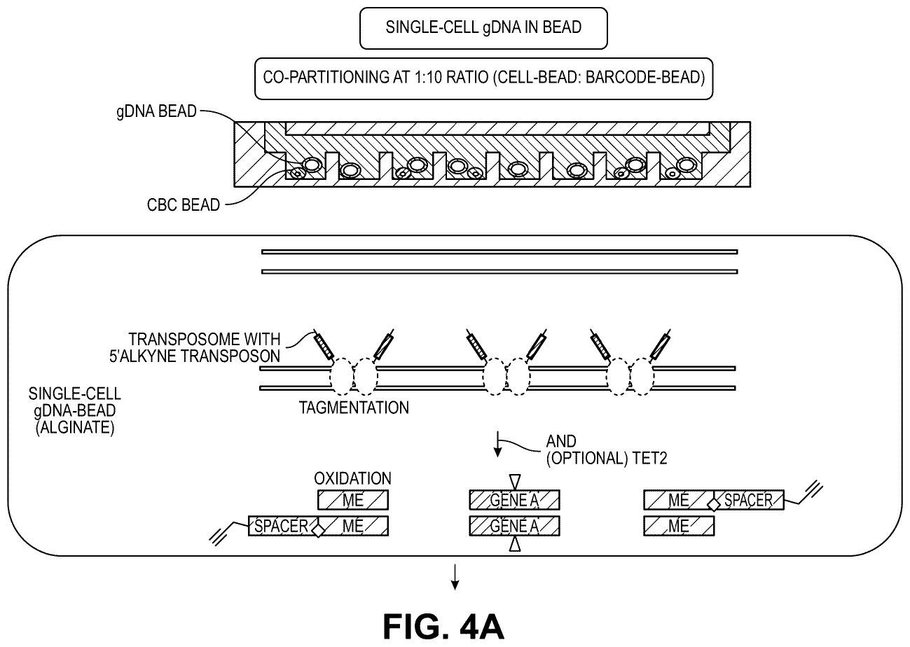

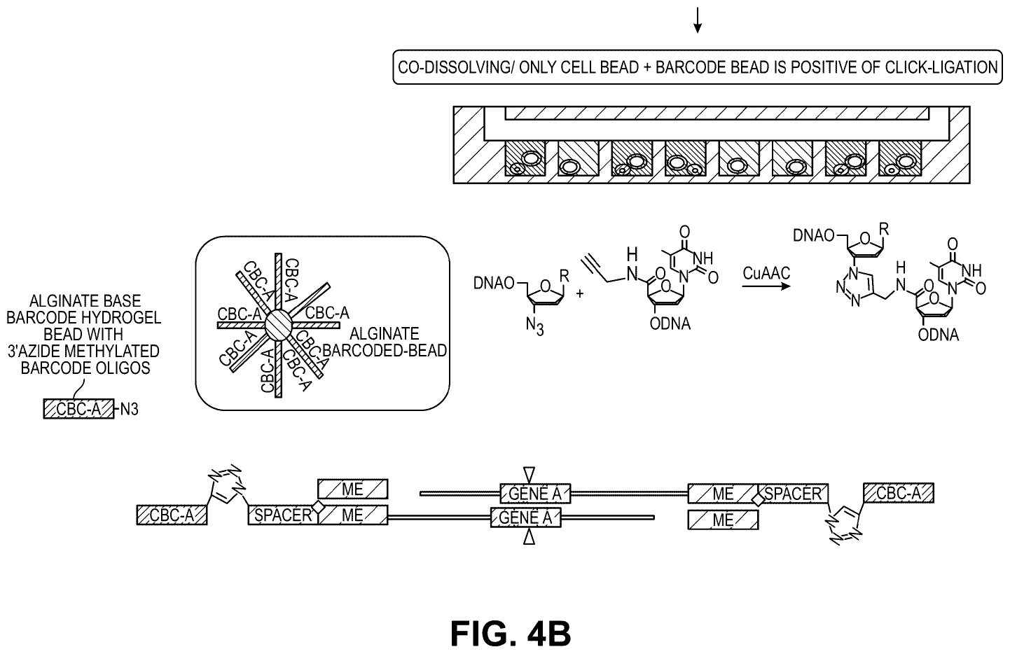

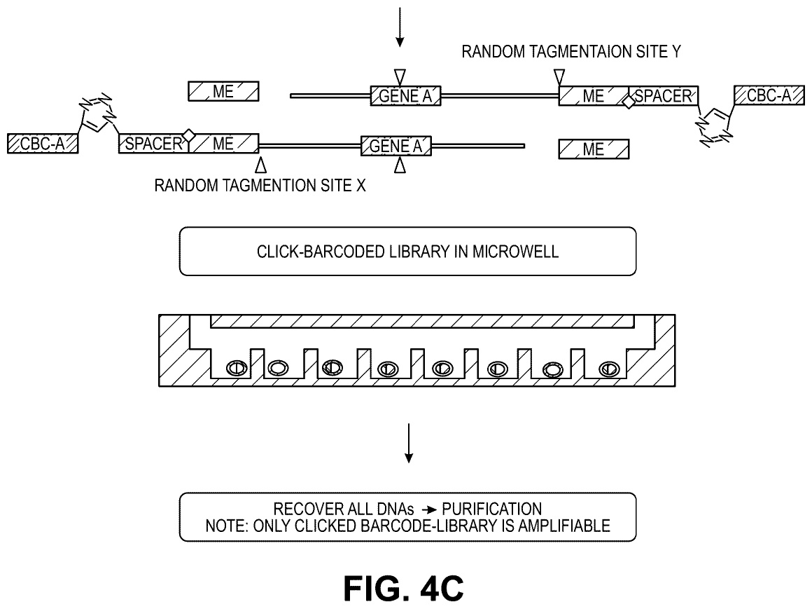

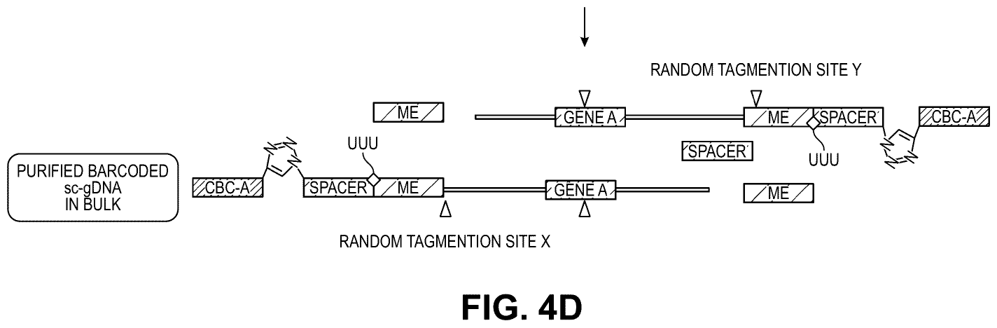

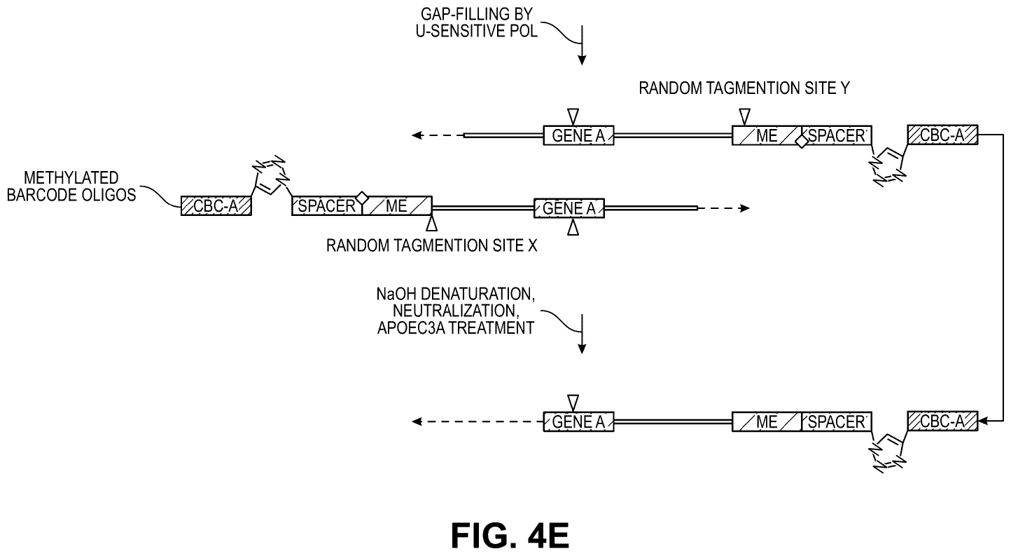

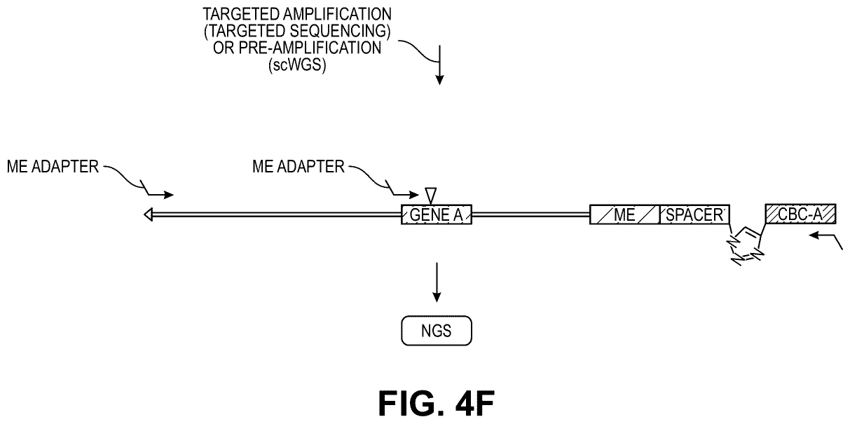

The products of the transposase-based fragmentation are double-stranded DNA fragments comprising in a first strand: (i) a first 5′ end linked to a first adaptor oligonucleotide having a 5′ alkyne and (ii) a first 3′ end and in a second strand: (iii) a second 5′ end linked to a second adaptor oligonucleotide having a 5′ alkyne and (iv) a second 3′ end. See, e.g., FIG. 4A. In the methods described herein, both strands can subsequently be linked to the same bead-specific barcode using click chemistry. For example, DNA from a single cell can be delivered to a partition (e.g., a microwell) as either the intact cell or as a lysed cell delivered within a hydrogel bead as described herein and then combined in the same partition with a barcoding bead that is linked to a plurality of identical barcoding oligonucleotides having a 3′ end that reacts with the 5′ end of the DNA fragments through click chemistry. See, e.g., FIG. 4B. For example, if the 5′ ends of the DNA fragments have a 5′ alkyne and the 3′ ends of the barcoding oligonucleotides have a 3′ azide moiety, click chemistry catalysis (CuAAC) or promotion (SpAAC) can be used to link the DNA fragment strands to barcoding oligonucleotides. In some embodiments, this method results in identical barcodes on each strand of the DNA fragment.

In some embodiments, the adapter oligonucleotides include one or more (e.g., 2, 3, 4, or more) uracils or other non-natural nucleotides or a carbon spacer between the ME sequence and the 5′ sequence (referred to as a “spacer” in the figures and including a universal sequence as a PCR handle, for example). See, e.g., FIG. 4D, where the uracils are indicated by a diamond shape. As discussed elsewhere, once the barcoding oligonucleotides are linked to the end of the DNA fragments, a uracil (or non-natural nucleotide or a carbon spacer)-sensitive polymerase can be used to amplify the barcoded DNA fragments, thereby preventing concatemerization.

The barcoding oligonucleotides can be delivered by a bead, to which the oligonucleotides are linked. The bead will be attached to multiple copies of the same oligonucleotide, for example, at least about 10, 50, 100, 500, 1000, 5000, 10,000, 50,000, 100,000, 500,000, 1,000,000, 5,000,000, 10,000,000, 10⁸, 10⁹, 10¹⁰ or more copies of the same or substantially identical oligonucleotide can be attached to one (e.g., the same) bead. The barcoding oligonucleotides will comprise at least a bead-specific barcode sequence and (i) a 3′ end comprising an azide or alkyne allowing for click chemistry reactions as described herein or (ii) a capture sequence for annealing to a target sequence on the adapter oligonucleotides, depending on how the barcoding oligonucleotides are linked to the adapter on the DNA fragments.

Each oligonucleotide can be linked at its 5′ end or elsewhere on the oligonucleotide to the bead and in some embodiments can include a cleavable moiety to remove the oligonucleotides from the bead, e.g., before the oligonucleotides are linked to the DNA fragments comprising the adapter sequence. In some embodiments, the cleavable linker comprises a uridine incorporated site in a portion of a nucleotide sequence. A uridine incorporated site can be cleaved, for example, using a uracil glycosylase enzyme (e.g., a uracil N-glycosylase enzyme or uracil DNA glycosylase (UDG) enzyme). In some embodiments, the cleavable linker comprises a photocleavable nucleotide. Photocleavable nucleotides include, for example, photocleavable fluorescent nucleotides and photocleavable biotinylated nucleotides. See, e.g., Li et al., PNAS, 2003, 100:414-419; Luo et al., Methods Enzymol, 2014, 549:115-131. In some cases, the oligonucleotides are attached to bead through a disulfide linkage (e.g., through a disulfide bond between a sulfide of the solid support and a sulfide covalently attached to the 5′ or 3′ end, or an intervening nucleic acid, of the oligonucleotide). In such cases, the oligonucleotide can be cleaved from the solid support by contacting the solid support with a reducing agent such as a thiol or phosphine reagent, including but not limited to a beta mercaptoethanol, dithiothreitol (DTT), or tris(2-carboxyethyl)phosphine (TCEP).

The oligonucleotides from the bead will include, for example, a bead-specific barcode such that the bead-specific barcode sequence on a first oligonucleotide can be used to distinguish it from a bead-specific barcode from a second oligonucleotide from a different bead. The 3′ end of the oligonucleotides will comprise the reverse complement of one of the universal sequences from the adaptor sequences added to the fragments such that the oligonucleotides from the beads can be used as primers in a primer extension (e.g., PCR) reaction using one strand of the gap-filled hybrid molecule fragments having adaptor sequences at their ends as a template, or the 3′ end will have an appropriate click chemistry moiety (e.g., an azide or alkyne). See, e.g., FIG. 4B. In some embodiments, one or more nucleotide (e.g., cytosines) are methylated in the barcoding oligonucleotide.

The above methods of linking a transposase adapter oligonucleotide and a barcoding oligonucleotide through click chemistry can be used as desired. While certain workflows are described herein, the linking of a transposase adapter oligonucleotide and a barcoding oligonucleotide through click chemistry need not be used with the other steps described herein. Nevertheless, in some embodiments, it will be desirable to use the click chemistry linkage in the following methods.

In some embodiments, the methods and composition escribed herein comprise introduction of single cells into a hydrogel bead or SPC, wherein reagents can be later introduced into the beads via diffusion to lyse the cells within the beads, and in some embodiments allow for additional molecular reactions, for example, optionally reverse transcription to convert RNA from the cells to DNA, and tagmentation. In some embodiments, the cells comprising target nucleic acids are from a biological sample. In some embodiments, the sample comprises cells or target nucleic acids that are isolated from tissue or other biological samples. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc.

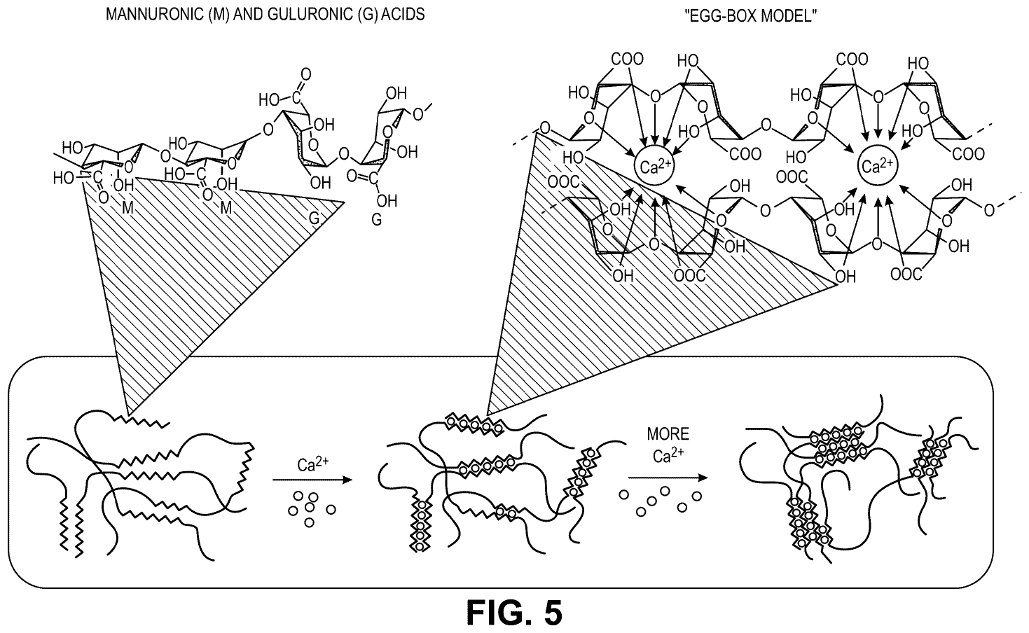

The hydrogels can be formed by encapsulating single cells. Formation of hydrogel beads around single cells can involve, for example, cell-encapsulated droplet generation by using oil to shear an aqueous phase with resuspended cells, and then bead formation by converting droplets to beads through gelation. See, e.g., Utech et al. Advanced healthcare materials 4.11 (2015): 1628-1633. In some embodiments, the hydrogel beads comprise alginate, which forms alginate matrices under increased calcium concentration. See, e.g., FIG. 5. Thus, single cells can be extruded in an alginate solution and be exposed to calcium, allowing for alginate matrices to form around single cells, thereby forming alginate hydrogel beads comprising single cells. In some embodiments, FACS or microfluidic-based sorting can be used to enrich cell-encapsulated beads.

An SPC is a capsule having a semi-permeable shell that allows for small molecules to pass through the shell while substantially retaining larger molecules, such as DNA (e.g., having at least 100 or at least 500 nucleotides), mRNA and optionally some proteins. For example WO-2023117364 describes SPCs with a semipermeable shells comprising a gel formed from a polyampholyte and/or a polyelectrolyte, wherein the polyampholyte and/or the polyelectrolyte in the gel is covalently cross-linked. In some embodiments, as described in WO-2023117364, the SPCs comprise an inner core in a liquid form, or in a hydrogel form and optionally enriched in polyhydroxy compounds belonging to the class of polysaccharides, oligosaccharides, carbohydrates, or sugars. For example, the core can comprise a polyhydroxy compound and/or an antichaotropic agent. In other embodiments, WO-2023099610 describes processes for manufacturing core-shell microcapsules and methods for using core-shell microcapsules to compartmentalize and optionally process biological entities and molecules. Other SPC formulations can comprise poly(ethylene glycol) diacrylate (PEGDA), for example as described in Michielin and Maerkl, Sci Rep. 2022; 12: 21391. Bomi et al., Langmuir 2015, 31, 6027-6034 describes yet other SPCs based on water-in-oil-in-water droplets that have a middle layer composed of photocurable resin and inert oil.

In some embodiments, following encapsulation of single cells into the hydrogel beads or SPCs, reagents can be diffused into the beads. The hydrogel bead or SPC will be selected to have a composition to substantially retain the nucleic acids of the cells while allowing for diffusion of reagents into the beads.

In some embodiments, reagents are diffused into the hydrogel beads or SPCs containing the cells, for example to fix and permeabilize the cells within. Exemplary fixative reagents that can be diffused into the hydrogel beads to the cells within can include the use of digitonin, or fixatives such as methanol (see, e.g., Alles, J., et al., BMC Biol 15, 44 (2017)), or paraformaldehyde. Permeabilization reagents can include, for example, Triton X-100.

In other embodiments, reagents that lyse the cells can be introduced into the hydrogel beads or SPCs. Exemplary lysis reagents can include, but need not be limited to, proteases or detergents or both.

In some embodiments, the genomic DNA from the lysed cells is contacted in the beads or SPCs with reagents to deplete nucleosomes present with the genomic DNA. Exemplary reagents that can deplete nucleosomes include, for example, one or more proteinase, for example, aspartic, glutamic, or metalloproteases, cysteine, serine, or threonine proteases, as well as for example proteinase K. Exemplary protein denaturation reagents include, e.g., SDS, NP40, Tween20, Triton, and Digitonin. In other embodiments, the cells will have intact chromatin such that some chromosomal regions are more accessible to the transposase than other chromosomal regions, allowing for ATACseq results to be generated. Exemplary conditions can include those for example, Nesterenko, et al., Proc. Natl. Acad. Sci. USA Vol. 118 No. 3 (2021).

In some embodiments, RNA from the cells is converted into DNA (i.e., cDNA), for example if a goal is to detect one or more cDNA from the cells. This can be achieved by diffusing into the hydrogel beads or SPCs sufficient reagents for revere transcription, e.g., a reverse transcriptase and nucleotides and a primer for initiating reverse transcription. Exemplary reverse transcription primers can include, for example, a polyA, random or gene-specific reverse transcription primer.

If methylation is also desired to be detected, for example in gDNA, the DNA in the hydrogels or SPCs can be contacted with an enzyme that converts methyl-cytosine into a different base while not changing unmethylated cytosine, or vice versa, and then treating the resulting products with an enzyme to convert one but not both classes of methylated or unmethylated cytosines. For example in some embodiments (e.g., when an enzyme has converted methyl-cytosine into a different base while not changing unmethylated cytosine), a DNA cytidine deaminase that deaminates cytosine, but not the converted methyl-cytosines, allowing one to distinguish differences in sequence reads. Optionally one can perform sequencing on samples that have and have not been treated with the above-described enzymes. In some embodiments, the tet methylcytosine dioxygenase 2 (TET2) enzyme is used to catalyze conversion of 5-methylcytosine to 5-hydroxymethylcytosine (5hmC) and then 5-carboxylcytosine (5caC) in the DNA fragments. Optionally a second enzyme, beta-glucosyltransferase, is also used to catalyze conversion of 5-methylcytosine to 5-hydroxymethylcytosine (5-hmC) residues and then beta-glucosyl-5-hydroxymethylcytosine (5gmC) in the DNA fragments.

The DNA in the beads or SPCs can then be contacted with a DNA cytidine deaminase (for example but not limited to APOBEC3). See, e.g., Schutsky, et al., Nucleic Acids Res. 2017 Jul. 27; 45(13): 7655-7665; Sun, et al., Non-destructive enzymatic deamination enables single molecule long read sequencing for the determination of 5-methylcytosine and 5-hydroxymethylcytosine at single base resolution BioRxiv December 2019; and Vaisvila, et al., Genome Res. 2021. 31: 1280-1289). This treatment results in enzymatic protection of 5-mC and/or 5-hmC prior to enzymatic deamination, for example by APOBEC3A. Subsequent sequencing allows for detection of both 5-mC and 5-hmC.

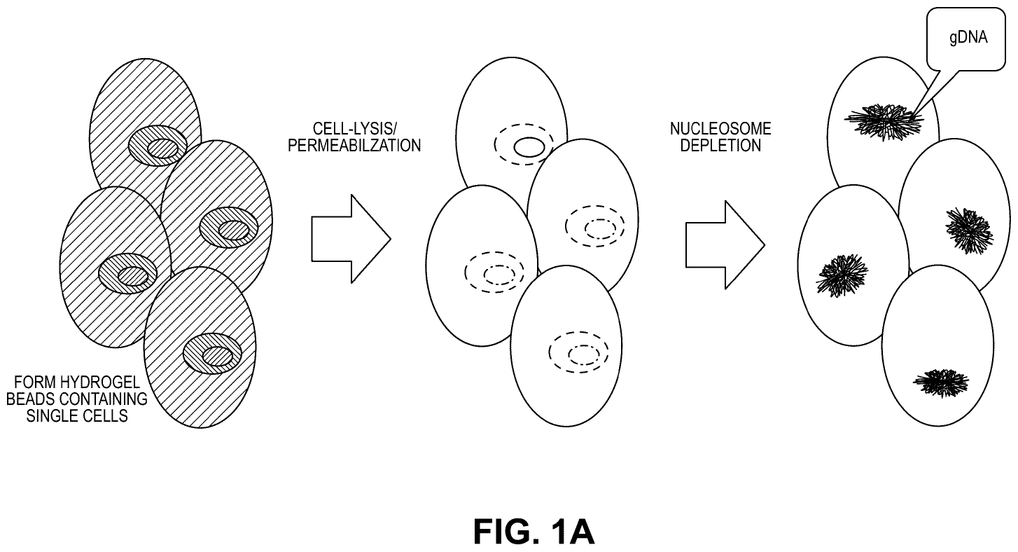

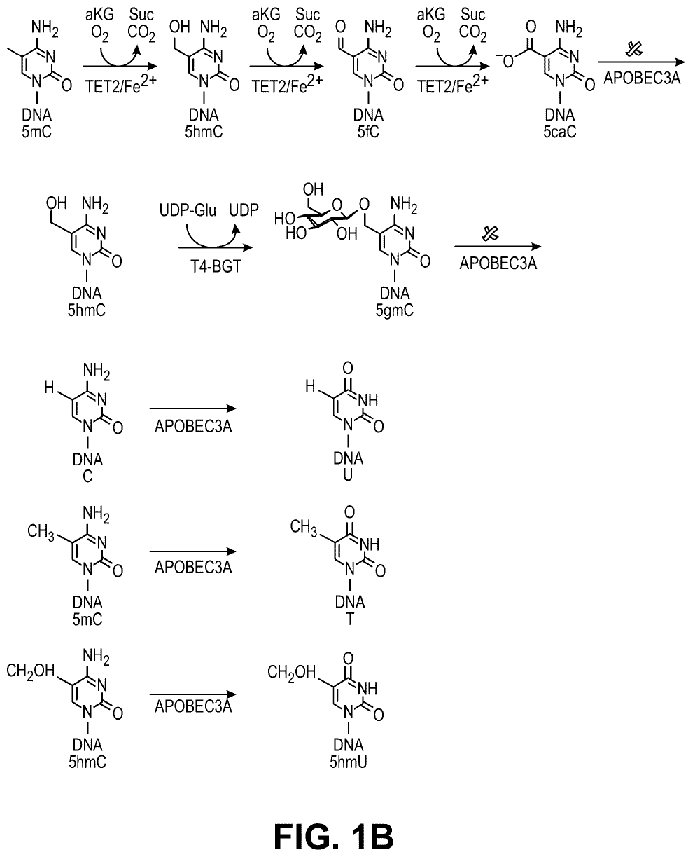

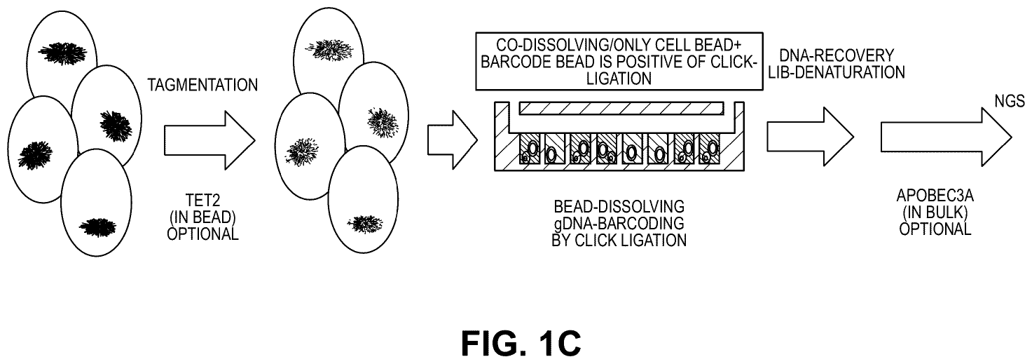

The DNA in the hydrogel beads or SPCs, whether treated to detect DNA methylation or not, can be, for example, genomic DNA, mitochondrial DNA, or cDNA. In any case, the DNA can subsequently be exposed in the hydrogel beads or SPCs to a transposase that fragments the DNA and adds adapter oligonucleotides to the 5′ ends of the DNA fragments as described elsewhere herein. See, e.g., FIG. 1C, item 4. Using a transposase to create DNA fragments and insert 5′ adapter oligonucleotides has been described elsewhere herein. The adapter-oligonucleotide loaded transposase will be diffused into the DNA in the hydrogel beads for sufficient time to generate DNA fragmentation of the size desired. The hydrogel bead will be sufficiently porous to allow the transposase to diffuse in, while the gDNA fragments will be too large to readily diffuse from the bead under the conditions used. Note the treatment of the DNA for methylation, if desired, can occur before or after contact with the transposase (tagmentation).

The hydrogel beads or SPCs comprising the tagmented DNA can subsequently be introduced into partitions. For example, conditions can be selected depending on the nature of the partitions to result in a majority of the hydrogel beads or SPCs being the only hydrogel bead or SPC containing same materials (e.g., sample DNA) in a particular partition, i.e., 1:1 sample hydrogel bead (or SPC):partition. In some embodiments, the partitions are wells, for example microwells. The size of the well as well as size of the mouth of the well can be used to control entry of the hydrogel beads or SPCs, resulting in a majority of beads or SPCs as the only sample hydrogel bead or SPC in the well. The partitions containing the hydrogel beads or SPCs comprising the tagmented DNA (referred to herein as “cell reaction hydrogel beads”) will also include a barcoding hydrogel bead linked to barcoding oligonucleotides that will subsequently be linked to the tagmented DNA. For example, the barcoding oligonucleotides will comprise (i) a barcode sequence that identifies the barcoding hydrogel bead and either or both of (ii) a 3′ azide or alkyne moiety (if click chemistry will be used to link to the tagmented DNA) or (iii) a 3′ end sequence (e.g., 2-10 bases or more) that anneals to the ends of the tagmented DNA. In some embodiments, the cell reaction hydrogel beads and barcoding hydrogel beads are introduced into the partitions at the same time (e.g., as a process of forming the partitions). In some embodiments, the cell reaction hydrogel beads are introduced after the barcoding hydrogel beads. In some embodiments, the cell reaction hydrogel beads are introduced before the barcoding hydrogel beads.

As noted above, in some embodiments, the cell reaction hydrogel beads or SPCs and barcoding hydrogel beads are introduced into wells (e.g., microwells). In some embodiments, the wells are provided in an array. In some embodiments, the wells of the array are sized such that the wells allow for one but only one cell reaction hydrogel bead and one barcoding hydrogel bead in a well. The solid supports are typically spherical (e.g., beads). However, in some embodiments, the size of the wells can be large enough to accept up to 2, 3, 4, 5, 6, 7, 8, 9, or 10 hydrogel beads per well. The wells of the array can be a combination of wells of different depths such that no more than a quantum (0, 1, 2, 3, 4, 5, etc.) number of beads of the same size can be accommodated in the microwells and the location for each type of microwell of a certain depth is pre-determined. Exemplary array of wells and well descriptions can be found for example in U.S. Pat. Nos. 9,103,754 and 10,391,493. The array of wells can include any suitable number of wells (e.g., on the scale of 100, 1,000, 10,000 wells, 50,000 wells, 100,000 wells, 1 million wells, 2 million wells, 3 million wells, 4 million wells, 5 million wells, 6 million wells, 7 million wells, 9 million wells, 10 million wells, 50 million wells, etc.).

The cell reaction hydrogel beads or SPCs and barcoding hydrogel beads can be introduced into wells wherein more barcoding hydrogel beads are introduced compared to cell reaction hydrogel beads or SPCs. In some embodiments, the cell reaction hydrogel beads or SPCs and barcoding hydrogel beads are delivered to the wells in a ratio of for example, 1:2 to 1:50, for example 1:5-1:20, for example 1:10. See, e.g., FIG. 4A.

Once the cell reaction hydrogel beads or SPCs and barcoding hydrogel beads are in the partitions, the hydrogels (and optionally SPCs) can be disrupted (e.g., dissolved or enzymatically digested) to release the adaptor-linked genomic DNA fragments from the cell reaction hydrogel beads or SPCs and to release the barcoding oligonucleotides from the barcoding hydrogel bead or SPC. In some embodiments, the hydrogels beads are formed from an alginate matrix and a calcium chelator is contacted to the beads in the wells to dissolve the alginate matrix. In some embodiments, the hydrogel beads or the SPCs are contacted with an enzyme to enzymatically digest the hydrogel beads or SPCs. As one example, the hydrogel beads or SPCs are composed of polysaccharides, e.g. dextran and/or gelatin, can be enzymatically degraded to digest these polysaccharides. Exemplary enzymes can include, but are not limited go, dextranase, gelatinases, and glycogenase. Subsequently, the DNA fragments can be linked to the barcoding oligonucleotides.

If the adaptor oligonucleotide introduced to the DNA fragments by the tagmentase comprises a 5′ alkyne moiety or azide moiety and the barcoding oligonucleotide has a corresponding azide or alkyne moiety, click chemistry linkage can be induced, for example upon introduction of a catalyst. For example, in some embodiments copper is added to the partition to induce Copper(I)-catalyzed azide-alkyne cycloaddition (CuAAC). Other click chemistry methods can alternatively be used to link the DNA fragments to the barcoding oligonucleotides.

In embodiments in which click chemistry is not employed, linkage can be achieved by annealing the adaptor-linked genomic DNA fragment to a barcoding oligonucleotide that can anneal to the adaptor portion of the fragments. These options can in some embodiments first comprise a gap filling step with a polymerase after tagmentation, allowing for formation of a second strand complementary to the adapter linked to a first DNA strand during tagmentation, and this second strand complementary to the adapter can be annealed to a reverse complementary sequence in the barcoding oligonucleotide, allowing the sequences to anneal. Subsequently, primer extension with a polymerase can be used to amplify the annealed nucleic acids, forming adaptor-linked genomic DNA fragments.

In the embodiments described above, the genomic DNA fragments become linked to the barcoding oligonucleotides. The contents of the wells can then be combined into a bulk mixture with nucleic acids from different wells comprising well (and generally cell)-specific barcodes, allowing for one to identify sequence reads from different wells (and cells).

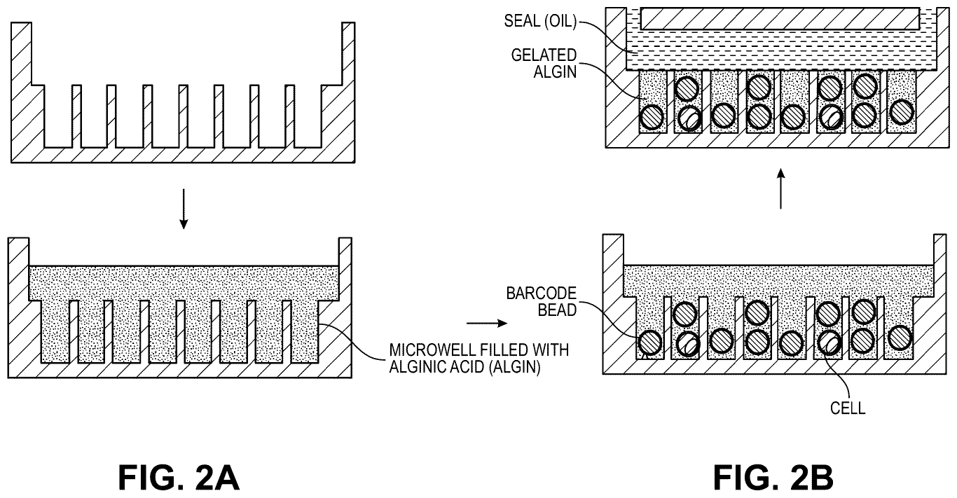

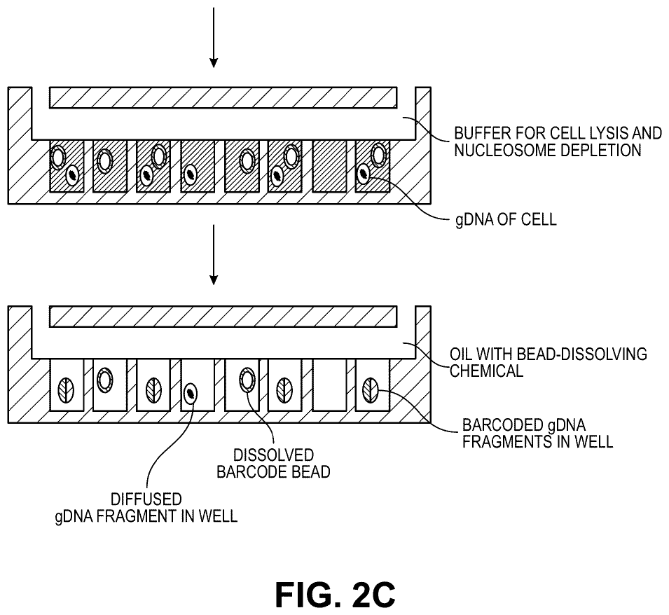

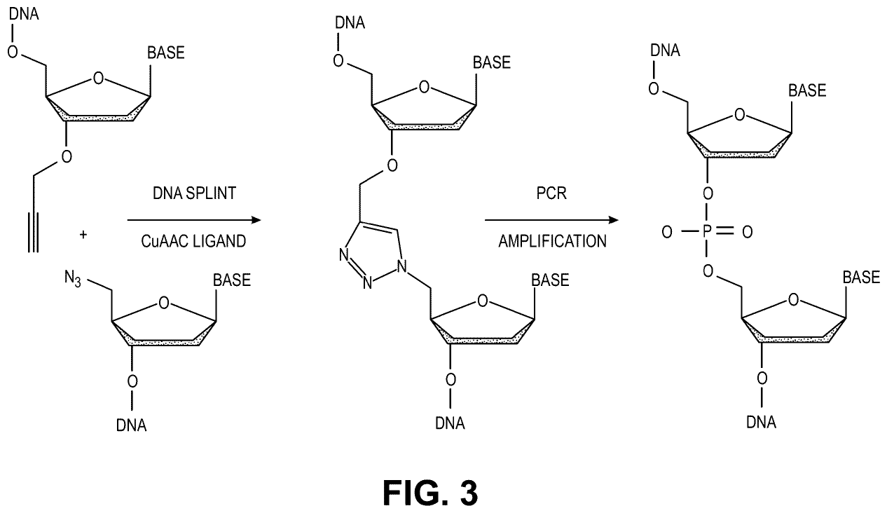

In some embodiments, as discussed above, the hydrogel beads or SPCs containing DNA fragments are delivered to wells, i.e., after cell lysis, tagmentation, etc. However, in an alternative embodiment, intact cells (e.g., optionally not encompassed by a hydrogel bead) are introduced into the wells along with barcoding hydrogel beads, preferably in a 1:1 ratio, e.g., a single cell and a single barcoding hydrogel bead in a well. The well can also contain alginic acid before (see FIG. 2A), or after the cell and barcoding hydrogel bead is introduced into the well. Once the alginic acid, cell and barcoding hydrogel bead are in the well, an alginate gel matrix can be induced (FIG. 2B), for example by introducing calcium into the wells (see, e.g., FIG. 5), thereby immobilizing the bead and cell in the well. Alginic acid, also called algin, is a naturally occurring, edible polysaccharide found for example in brown algae. It is hydrophilic and forms a viscous gum when hydrated. With metals such as sodium and calcium, its salts are known as alginates. See, e.g., Lee and Mooney, 2012, Prog. Polym. Sci. Prog Polym Sci. 2012 January; 37(1): 106-126. In some embodiments, an oil comprising acetic acid or another acid can be used to seal the wells. As a result, after well sealing, the lower pH environment breaks the bonding between calcium and EDTA. Free calcium will therefore cross-link the alginate ionically to form an alginate gel in well.

Once the cell is immobilized in the well or other partition, reagents can be diffused through the alginate matrix into the well, similar to what is described above in the context of diffusion to hydrogel beads as discussed above. See, e.g., FIG. 2C. For example, cell lysis reagents, cell permeabilization and fixation reagents, reagents to remove nucleosomal, or histone proteins from genomic DNA, reagents to form cDNAs from cellular RNA, or tagmentation reagents, other reagents as described above in the context of diffusion into hydrogel beads, can be diffused through the alginate matrix in the cells, thereby achieving the desired reactions to lyse the cell, or alternatively fix and permeabilize the cell, remove proteins associated with genomic DNA, form cDNAs, perform tagmentation to form adaptor-linked genomic DNA fragments or perform other reactions on the nucleic acids in the, optionally lysed or permeabilized, cells. Once the desired reactions are complete and adaptor-linked genomic DNA fragments have been formed, the alginate matrix can be dissolved. This can be achieved for example by contacting the alginate matrix with a calcium chelator. Exemplary calcium chelators include, for example, EDTA or sodium citrate. The contents of the wells can then be combined into a bulk mixture with nucleic acids from different wells comprising well (and generally cell)-specific barcodes, allowing for one to identify sequence reads from different wells (and cells).

Regardless of whether the workflows described above in the context of FIGS. 1A-C of 2A-C are employed, the resulting product can be a bulk mixture of DNA fragments from different cells, linked to barcoding oligonucleotides via click chemistry. The mixtures will therefore comprise DNA fragments comprising a first and a second strand, wherein the 5′ ends of the first and second strands comprise the adapter oligonucleotides introduced by the tagmentase (transposase) and in embodiments involving click chemistry, a click chemistry-caused linkage with a barcoding oligonucleotide. As explained herein, the adapter oligonucleotides comprise a mosaic end (ME) sequence and a second sequence, sometimes referred to herein as a “spacer” sequence, wherein the space sequence is at the 5′ end of the adapter oligonucleotide. In some embodiments, one or more nucleotides are present between the ME and spacer sequences that cannot be processed by certain polymerases. For example, certain polymerases are sensitive to the presence of uracils or other non-natural nucleotides or carbon spacers and will perform primer extension using uracils as a template. This in some embodiments, the one or more nucleotides are present between the ME and spacer sequences are one or more uracils, for example 1, 2, 3, 4 or more uracils. In some embodiments, a carbon spacer is between the ME and spacer sequences. Exemplary carbon spacers include but are not limited to 3C, 9C, 18C, where the number indicates the number of carbons. See, e.g., Wang et al., Bioorganic & Medicinal Chemistry Letters, Volume 18, Issue 12, 15 Jun. 2008, Pages 3597-3602. Exemplary products of the click chemistry linkage between the DNA fragments and barcoding oligonucleotides are depicted at the bottom of FIG. 4B and the top of FIG. 4C and in FIG. 4D (the latter showing one fragment of a bulk mixture of a plurality of fragments from multiple cells).

Optionally the barcoded DNA fragments can be purified in the bulk mixture. For purification, in some embodiments, DNA-binding magnetic beads are added to the microwells, which contains barcoded DNAs. After incubation, DNA-bound magnetic beads are removed by a magnet and then washed to remove carryover chemicals. After washing, purified DNAs that are bound to the magnetic beads are eluted for subsequent library preparation steps.

Subsequently, amplicons can be generated for next generation sequencing. In some embodiments, gap-filling of the first and second strands can subsequently be performed with a polymerase sensitive to the nucleotide or nucleotides between the spacer and ME sequences. Exemplary polymerases that stop or stall at uracils or non-standard linkers such as spacer carbon chains include but are not limited to archaeal DNA polymerase from Pyrococcus furiosus (Horvath et al., Nucleic Acids Res. 2010 November; 38(21): e196.) and Vent (Greagg et al., PNAS USA 96 (16) 9045-9050 (1999)). By gap-filling with such polymerases, extension stops at the sequence between the spacer and ME sequence, thereby generating extension products with a copied spacer and click-chemistry linked-barcode from the opposing strand. See, e.g., FIG. 4E. Strands can subsequently be denatured (e.g., using base, for example NaOH, and/or heat and neutralized if needed). In embodiments in which) tet methylcytosine dioxygenase 2 (TET2) that catalyzes conversion of 5-methylcytosine to 5-hydroxymethylcytosine (5hmC) and then 5-carboxylcytosine (5caC) in the DNA fragments or (ii) a beta-glucosyltransferase that catalyzes conversion of 5-methylcytosine to 5-hydroxymethylcytosine (5-hmC) residues and then beta-glucosyl-5-hydroxymethylcytosine (5gmC) in the DNA fragments at an earlier stage, the denatured strands can be contacted with a DNA cytidine deaminase that deaminates cytosine but not 5caC or 5gmC. An exemplary DNA deaminase is APOBEC3A. This will allow for differentiation of methylated and unmethylated cytosines in the original DNA sample.

Following primer extension as described above, the extension product can be amplified using a primer, for example either specific for a target sequence or that anneals to the ME sequence in the extension product. The former is useful where a specific target sequence is to be sequenced whereas the latter is useful for whole genome sequencing analysis. Once the amplification products are firmed, they can be applied to sequencing workflows.

The amplicons can be sequenced by any nucleotide sequencing technology desired. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

In some embodiments, the methods described herein comprise contacting the cells with one or more oligonucleotide-labeled binding agent, wherein the binding agent oligonucleotide comprises a binding agent-specific barcode sequence and the appropriate moiety at its 5′ or 3′ end to allow for use of click chemistry to later link the binding agent oligonucleotide to a barcoding oligonucleotide, thereby allowing one to determine from which cell the antibody bound, based on the barcoding oligonucleotide sequence. Thus, in some embodiments, the methods are a variant of an Abseq method. Abseq methods (see, e.g., Mair et al., Cell Rep. 2020 Apr. 7; 31(1):107499), which can be used with the click chemistry methods described herein, use specific antibodies to detect epitopes of interest. Antibodies are labeled with sequence tags that can be read out with DNA sequencing. This method can be used to sequence-tag both surface, intracellular proteins, or both, of different cell types at the single-cell level and distinguishing between the cells by their protein expression profiles. In some embodiments of the methods described herein, the antibody oligonucleotide comprises a 5′ alkyne moiety that can be later linked to a 3′ azide moiety on the barcoding oligonucleotide. In some embodiments, the cells can be contacted with a binding agent before cell lysis and the linking of the binding agent oligonucleotide to the barcoding oligonucleotide occurs, for example, when the DNA fragments are also linked to the barcoding oligonucleotides (thereby generating partition-specific barcoded DNA fragments and partition-specific barcoded binding agent oligonucleotides). The cells can be contacted with the binding agents before or after the hydrogel beads is formed around the cells. The sequencing can comprise sequencing the partition-specific barcoded binding agent oligonucleotides, allowing one to associate cell binding by the binding agents to specific cells, as represented by the barcoding oligonucleotide sequence. In some embodiments, the binding agents bind to surface antigens on the cells. In some embodiments, the cells are permeabilized and the binding agents bind to antigens in the cells.

Different binding agents can be bound to the cells, where binding agents having different binding affinities have different binding agent barcodes, allowing for tracking of multiple binding agents specificities for different single cells.

Examples of binding molecules include but are not limited to an antibody or an aptamer. See, e.g., Stoekius, et al., Genome Biology 19:224 (2018); Delley, et al., bioRxiv 1-10 (2017).

### EXAMPLES

### Example 1

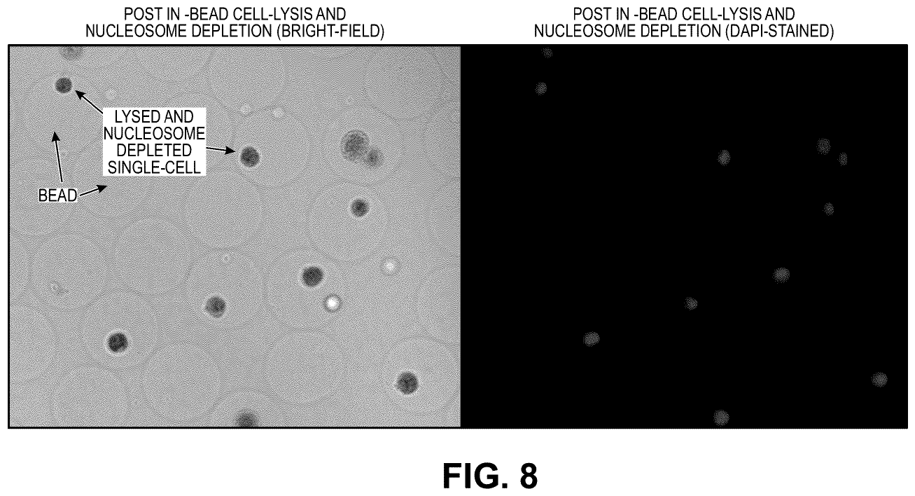

The cell morphology and gDNA accessibility of single cell encapsulated hydrogel beads were evaluated under the microscope. Human K562 single-cell encapsulated hydrogel beads were generated by a microfluidic device and then washed with culture medium before in-bead cell-lysis and nucleosome depletion processes. Cells encapsulated in hydrogel beads were stained with DAPI (4′,6-diamidino-2-phenylindole), a blue-fluorescent DNA stain, to reveal the accessibility of genomic DNA after the in-bead cell-lysis/nucleosome depletion treatment. Bright-field (left) and DAPI-stained (right) images of single-cell encapsulated hydrogel beads were captured by ZOE fluorescent cell imager as shown in FIG. 8.

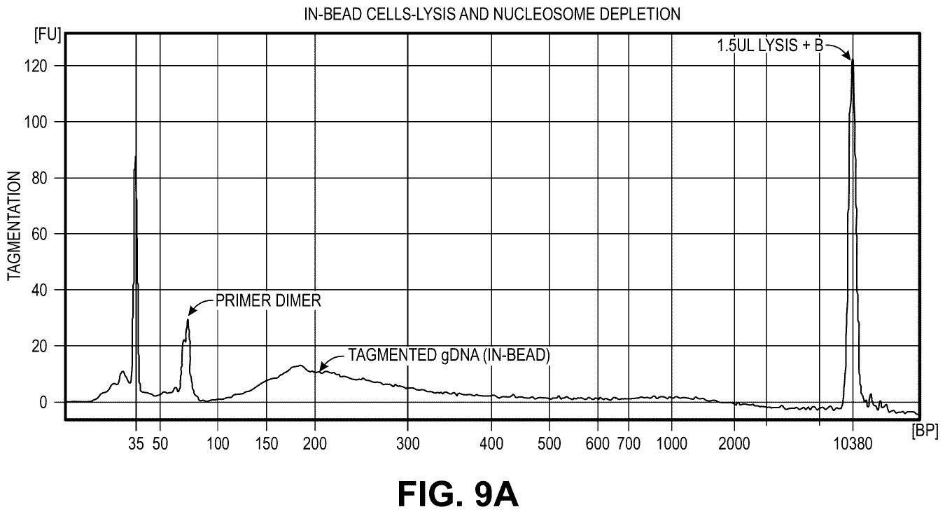

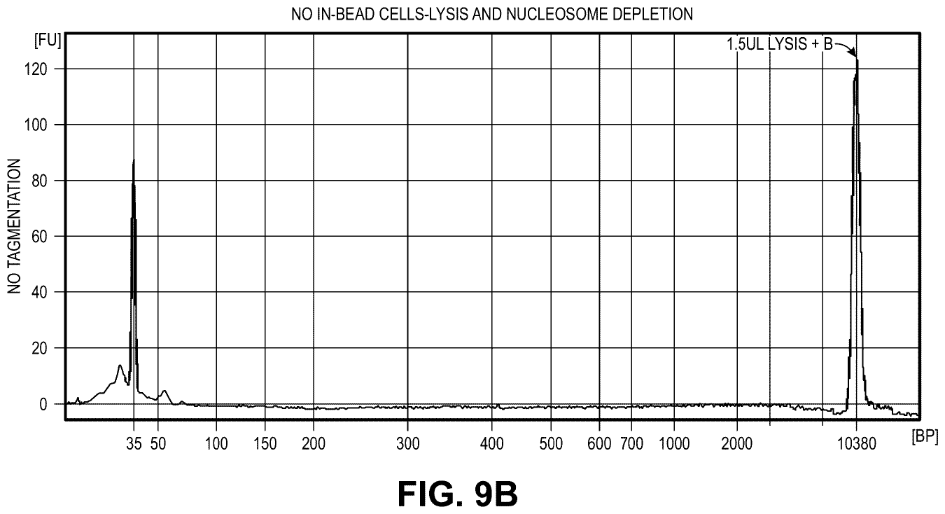

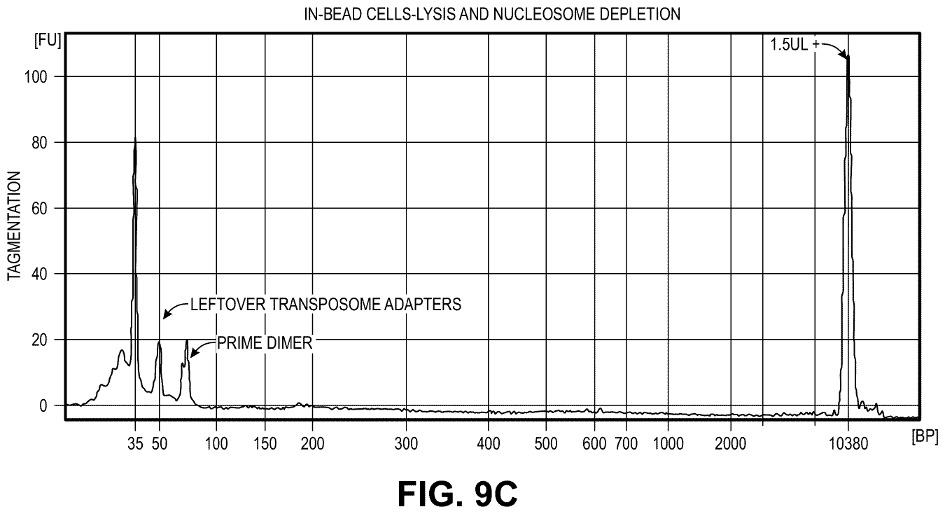

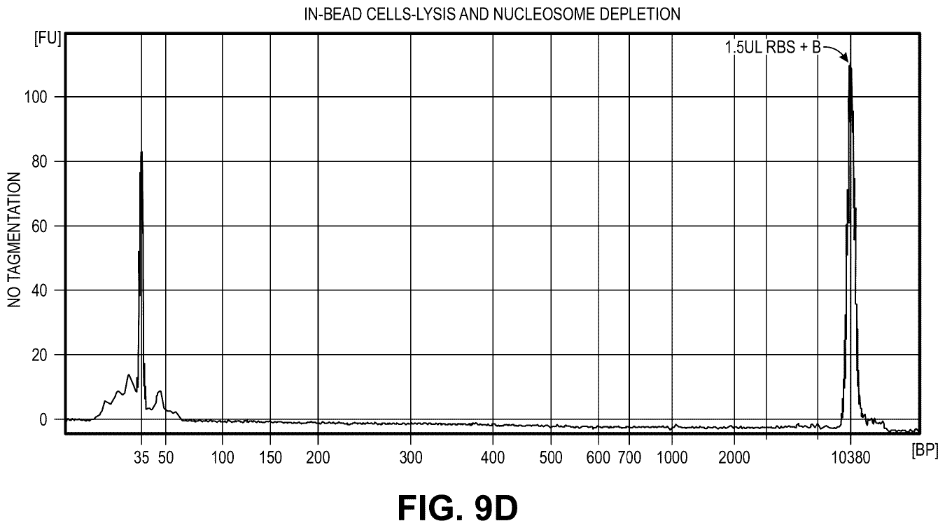

Human K562 single cells encapsulated in hydrogel beads were generated by a microfluidic device and then washed with culture medium before in-bead cell-lysis/nucleosome depletion, and tagmentation processes. FIGS. 9A-9D show the size profile of tagmented K562 cell human gDNA fragments from single cells encapsulated in hydrogel beads. K562 cells encapsulated in beads were incubated in Proteinase K lysis buffer (A and B) or PBS (C and D) for 20 minutes with orbital shaking. After incubation and bead washing, hydrogel beads were incubated at 55 degrees C. with (A and C) or without (B and D) transposase treatment. Following the in-bead tagmentation process, the hydrogel beads were dissolved and the tagmented DNAs were retrieved and purified for size profiling by using BioAnalyzer.

### Example 2

### Methodology:

Single human cell encapsulated alginate beads were generated by using the methodology described here. Cells were lysed in-bead and then the naked single-cell gDNA was tagmented as described previously. Following in-bead tagmentation, single-cell encapsulated beads were dispensed into wells of 96-well plate so that each well contains only one cell. Bead was then digested and the DNA was indexed and then recovered from the wells. Libraries of 4 individual wells (i.e. 4 individual cells) were pooled for next-generation sequencing.

The DNAseq genome coverage of individual cells was evaluated by using MAD (Median Absolute Deviation) score as described by Adey A. C et al. High-content single-cell combinatorial indexing. Nature Biotechnology. 39, 1574-1584 (2021). In brief, after deduplication and mapping against the hg38 reference human genome, unique sequencing reads within every 500 kb genomic intervals (i.e. bins) were counted for MAD scoring. MAD score, a robust dispersion measurement of outliers, is a statistic method of describing the reads distribution across bins assigned to the human genome. It computes the median value of read counts across bins, and then calculate how far each value is from the median of all values to determine relative coverage of reads to the human genome.

### Results

Table 1 shows the median and standard deviation of MAD score of the DNAseq libraries of four individual cells generated by using this method as compared to the value of the single-cell DNAseq assay from Adey A. C et al. High-content single-cell combinatorial indexing. Nature Biotechnology. 39, 1574-1584 (2021).

TABLE 1
Evaluation of sample's coverage
uniformity measured by MAD score

| | MAD score of single-cell | MAD score of single-cell |
|---|---|---|
| | DNAseq assay using this | DNAseq assay from Adey's |
| | invention | publication |
| Cell_1 | 0.225 | n/a |
| Cell_2 | 0.210 | n/a |
| Cell_3 | 0.211 | n/a |
| Cell_4 | 0.237 | n/a |
| Mean ± | 0.221 ± 0.013 | 0.152 ± 0.025 and 0.219 ± 0.041 |
| STD | | |

Although the foregoing disclosure has been described in some detail by way of illustration and example for purposes of clarity of understanding, one of skill in the art will appreciate that certain changes and modifications may be practiced within the scope of the appended claims. In addition, each reference provided herein, including patents, patent applications, non-patent literature, and Genbank accession numbers, is incorporated by reference in its entirety to the same extent as if each reference was individually incorporated by reference. Where a conflict exists between the instant application and a reference provided herein, the instant application shall dominate.

What is claimed is:

1. A method of nucleotide sequencing, the method comprising forming cell reaction (i) hydrogel beads or (ii) semi-permeable capsules (SPCs) comprising single cells;
lysing the cells in the cell reaction hydrogel beads or SPCs such that at least a majority of nucleic acids of the cells is retained in the cell reaction hydrogel beads or SPCs, wherein the nucleic acids are DNA from the cell or RNA, and optionally converting the RNA into DNA with a reverse transcriptase;
contacting the DNA of the cells in the cell reaction hydrogel beads or SPCs with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked DNA fragment having the 5′ alkyne moieties;
partitioning in microwells the cell reaction hydrogel beads or SPCs comprising the DNA fragments with a barcoding hydrogel bead linked to barcoding oligonucleotides comprising (i) a barcode sequence that identifies the barcoding hydrogel bead and (ii) a 3′ azide moiety, thereby forming microwells containing one of the cell reaction hydrogel beads or SPCs and one of the barcoding hydrogel beads;
disrupting (e.g., dissolving) the cell reaction hydrogel beads or SPCs and barcoding hydrogel beads in the microwells;
after the disrupting, linking the 5′ alkyne moieties of the adaptor-linked DNA fragments to the 3′ azide moiety of the barcoding oligonucleotides via click chemistry to form a first and second barcoded strand of barcoded double-stranded DNA fragments, recovering barcoded DNA fragments from the microwells and forming a mixture of barcoded DNA fragments from different microwells; and
performing nucleotide sequencing of the mixture of barcoded DNA fragments.

2. The method of claim 1, wherein the DNA is genomic DNA or mitochondrial DNA.

3. The method of claim 2, wherein the DNA is genomic DNA and the method further comprises depleting nucleosomal or histone proteins from the lysed cells before the contacting.

4. The method of claim 1, wherein the nucleic acids are RNA and the method comprises converting the RNA into DNA with a reverse transcriptase.

5. The method of claim 1, further comprising, after the contacting and before the partitioning, contacting the DNA fragments in the hydrogel beads or SPCs with (i) a tet methylcytosine dioxygenase 2 (TET2) that catalyzes conversion of 5-methylcytosine to 5-hydroxymethylcytosine (5hmC) and then 5-carboxylcytosine (5caC) in the DNA fragments or (ii) a beta-glucosyltransferase that catalyzes conversion of 5-methylcytosine to 5-hydroxymethylcytosine (5-hmC) residues and then beta-glucosyl-5-hydroxymethylcytosine (5gmC) in the DNA fragments; and
after the forming, contacting the barcoded DNA fragments with a DNA cytidine deaminase that deaminates cytosine but not 5caC or 5gmC.

6. The method of claim 5, wherein the DNA cytidine deaminase is APOBEC3A.

7. The method of claim 1, wherein the cell reaction hydrogel beads, the barcoding hydrogel beads, or both, comprise cross-linked alginate.

8. The method of claim 7, wherein the dissolving comprises contacting the cross-linked alginate with a calcium chelator.

9. The method of claim 3, wherein the depleting of nucleosomal proteins from the lysed cells comprises contacting genomic DNA from the lysed cells with a protease, a detergent, or both a protease and a detergent.

10. The method of claim 1, wherein between the partitioning and the dissolving, sealing the microwells from each other with a water-impermeable barrier.

11. The method of claim 10, wherein the sealing comprises applying a layer of oil to cover the microwells.

12. The method of claim 1, wherein the performing nucleotide sequencing of the mixture comprises nucleotide sequencing of the first and second barcoded strand of barcoded genomic double-stranded DNA fragments.

13. The method of claim 1, wherein the first strand of the adaptor oligonucleotides comprises 5′-3′: a spacer sequence, one or more uracil or modified bases or carbon spacer and a transposase binding (ME) sequence and
further comprising amplifying the first and/or second barcoded strand of barcoded genomic double-stranded DNA fragments with a polymerase that stops primer extension at the one or more uracil or modified bases or carbon spacer to form a truncated amplicon.

14. The method of claim 1, further comprising amplifying the first and/or second barcoded strand of barcoded genomic double-stranded DNA fragments or the truncated amplicon with a first primer that anneals to the ME sequence.

15. The method of claim 14, wherein the amplifying further comprises amplifying the first and/or second barcoded strand of barcoded genomic double-stranded DNA fragments or the truncated amplicon with a second primer that anneals to the first strand of the adaptor oligonucleotide such that a resulting amplification product comprises the barcode sequence.

16. The method of claim 1, further comprising, before the lysing, contacting the cells with one or more different antibodies, wherein each antibody is linked to an antibody oligonucleotide comprising an antibody barcode sequence specific for the antibody and a 5′ alkyne moiety, and
wherein the linking further comprises linking the 5′ alkyne moiety on the antibody oligonucleotide to the 3′ azide moiety of the barcoding oligonucleotides via click chemistry to form a DNA molecule comprising the antibody-barcode and barcode sequence that identifies the barcoding hydrogel bead; and
nucleotide sequencing of DNA molecules comprising the antibody-barcode and barcode sequence that identifies the barcoding hydrogel bead.

17. The method of claim 16, wherein the contacting of the cells with the one or more different antibodies occurs before the forming.

18. The method of claim 16, wherein the contacting of the cells with the one or more different antibodies occurs after the forming.

19. A method of nucleotide sequencing, the method comprising,
providing a plurality of microwells containing alginic acid;
introducing into the microwells (i) single cells and (ii) barcoding hydrogel beads linked to barcoding oligonucleotides comprising (i) a barcode sequence that identifies the barcoding hydrogel bead and (ii) a 3′ azide moiety;
inducing gelation of the alginate to form an alginate matrix surrounding the cells in the microwells;
diffusing into the microwells reagents that lyse the cells, thereby releasing nucleic acids from the cells, wherein the nucleic acids are DNA from the cell or RNA, and optionally converting the RNA into DNA with a reverse transcriptase;
contacting the DNA of the lysed cells with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked genomic DNA fragment having the 5′ alkyne moieties;
dissolving the alginate matrix and the barcoding hydrogel beads in the microwells;
linking the 5′ alkyne moieties of the adaptor-linked DNA fragments to the 3′ azide moiety of the barcoding oligonucleotides via click chemistry to form a first and second barcoded strand of barcoded genomic double-stranded DNA fragments;
recovering barcoded DNA fragments from the microwells and forming a mixture of barcoded DNA fragments from different microwells; and
performing nucleotide sequencing of the mixture of barcoded DNA fragments.

20. A method of barcoding DNA, the method comprising
contacting DNA with a transposase that introduces breaks in the DNA to form a double-stranded DNA fragment and inserts adaptor oligonucleotides at the breaks, wherein the adaptor oligonucleotides comprise a first strand and a second strand, wherein 3′ ends of the first strand of the adaptor oligonucleotides are covalently linked to 5′ ends of each strand of double-stranded DNA fragment, and wherein the first strand of the adaptor oligonucleotide comprises a 5′ alkyne moiety, thereby forming an adaptor-linked DNA fragment having the 5′ alkyne moieties;
mixing the DNA fragments, optionally from a single cell, with a barcoding bead linked to barcoding oligonucleotides comprising (i) a barcode sequence that identifies the barcoding bead and (ii) a 3′ azide moiety; and
linking the 5′ alkyne moieties of the adaptor-linked DNA fragments to the 3′ azide moiety of the barcoding oligonucleotides via click chemistry to form a first and second barcoded strand of barcoded double-stranded DNA fragments, thereby barcoding the DNA.


SEQUENCE LISTING (6 sequences)

SEQ ID NO: 1 - DNA, 14 bp, synthetic construct
  source 1..14: mol_type = other DNA
tcgtcggcagcgtc

SEQ ID NO: 2 - DNA, 15 bp, synthetic construct
  source 1..15: mol_type = other DNA
gtctcgtgggctcgg

SEQ ID NO: 3 - DNA, 19 bp, synthetic construct
  source 1..19: mol_type = other DNA
agatgtgtataagagacag

SEQ ID NO: 4 - DNA, 33 bp, synthetic construct
  source 1..33: mol_type = other DNA
tcgtcggcagcgtcagatgtgtataagagacag

SEQ ID NO: 5 - DNA, 34 bp, synthetic construct
  source 1..34: mol_type = other DNA
gtctcgtgggctcggagatgtgtataagagacag

SEQ ID NO: 6 - DNA, 19 bp, synthetic construct
  source 1..19: mol_type = other DNA
ctgtctcttatacacatct